(19) United States
(12) Patent Application Publication
(10) Pub. No.: US 2021/0363592 A1
(43) Pub. Date: Nov. 25, 2021

(54) Detection of Signatures in a Breast Cancer Subject

(71) Applicant: Vanderbilt University, Nashville, TN (US)

(72) Inventors: Justin M. Balko, Nashville, TN (US); Margaret L. Axelrod, Nashville, TN (US)

(21) Appl. No.: 17/199,362

(22) Filed: Mar. 11, 2021

Related U.S. Application Data
(60) Provisional application No. 62/988,316, filed on Mar. 11, 2020.

(51) Int. Cl. C12Q 1/6886

(52) U.S. Cl. CPC C12Q 1/6886; C12Q 2600/158; C12Q 2600/112

(57) ABSTRACT

Methods of treatment for a subject having breast cancer, and who has received neoadjuvant chemotherapy (NAC), involve detecting expression levels of genes in a first signature including: PDCD1, NKG7, LAG3, GZMH, GZMB, GNLY, FGFBP2, and HLA-DRB5, and administering additional chemotherapy prior to surgery, or administering additional chemotherapy after surgery when the subject is identified as having a likelihood of residual disease (RD); or proceeding with surgery without administering additional chemotherapy when the subject is identified has having a likelihood of pathological complete response (pCR).

### RELATED APPLICATIONS

This application claims priority from U.S. Provisional Application Ser. No. 62/988,316 filed Mar. 11, 2020, the entire disclosure of which is incorporated herein by this reference.

### GOVERNMENT INTEREST

This invention was made with government support under grant number W81XWH1810149/BC170037 awarded by the Department of Defense. The government has certain rights in the invention.

### TECHNICAL FIELD

The presently-disclosed subject matter generally relates to treatment of breast cancer. In particular, certain embodiments of the presently-disclosed subject matter relate to predicting whether a subject who has received neoadjuvant chemotherapy will benefit from proceeding with a surgery and/or other therapy.

### INTRODUCTION

Patients with breast cancer are often treated with chemotherapy prior to surgery, which is referred to as neoadjuvant chemotherapy (NAC). NAC is sometimes provided in combination with immunotherapy. For example, anti-PD-L1 immunotherapy in combination with nab-paclitaxel has been approved for metastatic triple-negative breast cancer (TNBC).¹ Furthermore, addition of the anti-programmed death-1 (PD-1) monoclonal antibody pembrolizumab to neoadjuvant chemotherapy (NAC) can significantly enhance TNBC pathological complete response (pCR) rates².

Thus, existing clinical data indicate that chemotherapy combinations with immunotherapy demonstrate enhanced efficacy compared to chemotherapy alone. However, these results suggest a growing need to better understand how chemotherapy modulates the tumor-immune microenvironment (TIME).

High levels of stromal tumor-infiltrating lymphocytes (sTILs) in a pre-treatment biopsy are predictive of pCR in TNBC patients treated with NAC.³ In NAC-treated TNBC patients with residual disease (RD) at surgery or in untreated primary TNBC tumors, higher sTILs in the resected tumor also confer improved prognosis⁴⁻⁷.

However, uncertainty in connection with the immunomodulatory effect of chemotherapy on sTILs in patients, as well as the impact of chemotherapy on TIME, raises questions about the efficacy of sTTILS as a marker for anti-timer immunity in patients who have received NAC or NAC in combination with immunotherapy.

Following NAC, at the time planned for surgery, some patients are identified as having a pathological complete response (pCR). For pCR patients, because no tumor cells remain, one might conclude that surgery is unnecessary. Nonetheless, it is often the practice to perform surgery remove the ‘tumor scar’ from the patient. These pCR patients have a very good outcome (low chance of recurrence).

Following NAC, at the time planned for surgery, some patients are identified as having residual disease (RD). For RD patients, surgery is performed to extract the remaining tumor. However, despite the tumor extraction, the RD patients are more likely to have a recurrence.

Identifying patients at risk of recurrence, particularly in those with RD, is a major challenge. If patients could be included or excluded from having a risk of recurrence or RD, recommendations for further treatment or cessation of treatment could be appropriately provided. Patients who could benefit from surgery and/or further treatment could be identified, patients with a low-risk of recurrence could have additional piece of mind after surgery, and patients identified as having a pCR could ultimately be spared unproductive surgery.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes methods detecting expression of a combination of genes in a sample from a subject having breast cancer and who has received neoadjuvant chemotherapy (NAC), methods of determining likelihood of residual disease (RD) or pathological complete response (pCR), and methods of providing recommendations for further treatment or cessation of treatment could. The presently-disclosed subject matter provide methods, whereby subjects who could benefit from surgery and/or further treatment could be identified, and subjects identified as having a pCR could ultimately be spared unproductive surgery.

The method as disclosed herein is envisioned for use in connection with a subjecting having breast cancer. In some embodiments, the subject has triple-negative breast cancer (TNBC). The method as disclosed herein is also envisioned for use in connection with a subject who has received neoadjuvant chemotherapy (NAC). In some embodiments, it is possible that the subject will be consider surgery or other additional treatment.

In some embodiments of the presently-disclosed subject matter the method involves obtaining or having obtained a biological sample from the subject; and detecting or having detected expression levels in the sample of genes in one or more signatures.

Some embodiments make use of a first signature, sometimes referred to herein as a cytotoxic signature or a residual disease (RD) signature, includes the following eight genes PDCD1, NKG7, LAG3, GZMH, GZMB, GNLY, FGFBP2, and HLA-DRB5. In some embodiments of the method, at least five genes of considered.

Some embodiments of the method include a step of calculating a first signature score by adding the expression level of each of the genes selected for detection from PDCD1, NKG7, LAG3, GZMH, GZMB, GNLY, and FGFBP2; and subtracting the expression level of HLA-DRB5, if detected. The expression level can be expressed, for example, transcript count for the gene(s) being detected. In some embodiments, the method further involves identifying the subject as having a likelihood of residual disease (RD) when the first signature score is greater than a standardized control; or identifying the subject as having a likelihood of pathological complete response (pCR) when the first signature score is less than a standardized control. The standardized control can be selected according to methods know to those skilled in the art, for example, by detection of normalization genes.

Some embodiments of the method include a step of identifying the subject as having a likelihood of residual disease (RD) and/or cancer recurrence when there is an elevated level of each of the genes selected for detection from PDCD1, NKG7, LAG3, GZMH, GZMB, GNLY, and FGFBP2, and a reduced level of HLA-DRB5, if detected. Some embodiments of the method include a step of identifying the subject as having a likelihood of pathological complete response (pCR) when there is a reduced level of each of the genes selected for detection from PDCD1, NKG7, LAG3, GZMH, GZMB, GNLY, and FGFBP2, and an elevated level of HLA-DRB5, if detected.

Some embodiments of the presently-disclosed subject matter involve a second signature, sometimes referred to herein as a IFN/complement signature or pathological complete response (pCR) signature. The second signature includes the following sixty genes: SERPING1, IFIT3, IFI44L, IFI44, LAP3, FCGR1A, EPSTI1, IFIT2, TNFSF10, WARS1, IFITM3, MX1, MT2A, BATF2, IL15, IFIT1, STAT1, GBP4, ISG15, OAS3, JAK2, VAMP5, FGL2, PLSCR1, OASL, SAMD9L, USP18, SECTM1, APOL6, PLA2G4A, UBE2L6, CFB, PSME2, OAS2, STAT2, PARP14, CASP1, IFI35, HLA-DMA, GCH1, CD86, IL15RA, DDX60, LATS2, BST2, NMI, IFIH1, CASP4, EIF2AK2, PARP9, GBP2, TENT5A, OAS1, C1QC, C1QA, C2, KYNU, MMP14, PDP1, and CASP10.

In some embodiments of the method, at least ten genes of the second signature are considered. For example, in some embodiments the following ten genes of the second signature are considered: C1QC, CASP10, JAK2, IL15, TNFSF10, C1QA, IFIT3, EPSTI1, PSME2, and LAP3. In some embodiments of the method, more than 10 or even all sixty of the genes of the second signature are considered.

Some embodiments of the method include a step of calculating a second signature score by adding the expression level of each of the genes selected for detection. The expression level can be expressed, for example, transcript count for the gene(s) being detected. In some embodiments, the method also involves identifying the subject as having a likelihood of residual disease (RD) when the second signature score is less than a standardized control; or identifying the subject as having a likelihood of pathological complete response (pCR) when the second signature score is greater than a standardized control.

In some embodiments, the method includes identifying the subject as having a likelihood of residual disease (RD) and/or cancer recurrence when there is a reduced level of each of the genes in the second signature that are selected for detection. In some embodiments, the method includes identifying the subject as having a likelihood of pathological complete response (pCR) when there is an elevated level of each of the genes in the second signature that are selected for detection.

Some embodiments of the method further include administering or recommending administration of additional chemotherapy prior to surgery and/or administration of additional chemotherapy after surgery when the subject is identified as having a likelihood of RD; or proceeding or recommending proceeding with surgery without administering additional chemotherapy when the subject is identified has having a likelihood of pCR.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1C include data showing immunologic changes in breast tumors after neoadjuvant chemotherapy. FIG. 1A: High levels of stromal tumor-infiltrating lymphocytes (sTILs) are associated with RFS (left; n=41) and OS (right; n=42) after surgery in TNBC. Patients are stratified based on post-NAC sTILs 5 30% or >30%, scored as recommended by the International TILs Working Group^{22,23}, according to the predefined cut point⁴. FIG. 1 B: Heatmap demonstrating gene expression patterns for 770 immune-related genes (NanoString Pan-Cancer Immune Panel) across all patients (TNBC and non-TNBC; n=83 total patients, 166 samples). FIG. 1C: Heatmap of gene expression patterns as detailed in panel B, instead depicting the change in expression of each gene in matched paired (pre- and post-NAC; n=83) samples. Red data points represent an upregulation, while blue data points represent a downregulation in the post-NAC residual disease compared to the pre-treatment diagnostic biopsy.

FIG. 2A-2B include data indicating that pre-NAC sTILs have minimal prognostic value in breast cancer patients with residual disease. FIG. 2A: Pre-NAC sTILs are not associated with RFS (left) or OS (right) after surgery in TNBC patients with residual disease (n=41 and 42 patients, respectively). Confirmed TNBC patients are stratified based on post-NAC sTILs 5 30% or >30%, scored as recommended by the International TILs Working Group22,23, according to the pre-defined cut point4. FIG. 2B: Pre-NAC sTILs are not associated with RFS (left) or OS (right) after surgery in unselected patients with residual disease (n=75 and 81 patients, respectively).

FIG. 3A-3B include identification of immune-associated genes associated with RFS and OS in TNBC after chemotherapy. FIG. 3A: Individual genes (changes pre- to post-NAC) were tested iteratively in a univariate cox-proportional hazards model for their association with RFS (left) or OS (right) after chemotherapy and surgery. Individual genes are colored for their statistical significance (red: nominal p-value<0.05; green: q-value (FDR)<0.10; black: not significant). Selected top genes are labeled but are limited in number for clarity. Genes with negative coefficients (left of the center line) are associated with better outcome, while genes with positive coefficients (right of the center line) are associated with worse outcome. FIG. 3B: Representative Kaplan-Meier plots for selected detrimental (CDH1; e-cadherin) and beneficial (CD70) genes are shown. Strata are defined by tertiles, and generally represent upregulation during NAC (blue), no change/equivocal (green), and downregulation (red). P-values represent the log-rank test for trend.

FIG. 4A-4B include data illustrating that the prognostic value of sTILs is primarily confined to the post-NAC specimen in TNBC patients with residual disease. FIG. 4A: Post-NAC sTILs are moderately associated with RFS (left), but not OS (right) after surgery in unselected patients with residual disease (n=74 and 80 patients, respectively). FIG. 4B: Post-NAC sTILs are not associated with RFS (left), or OS (right) after surgery in non-TNBC patients with residual disease (n=33 and 38 patients, respectively).

FIG. 5 includes data indicating that the change in sTILs during NAC is not prognostic for outcome in TNBC patients with residual disease. The change in sTILs from pre- to post-NAC is not associated with RFS (left) or OS (right) after surgery in TNBC patients with residual disease (n=41 and 42 patients, respectively). Strata are defined as whether sTILs was decreased/equivocal (red) or increased (blue).

FIG. 6A-6B include data showing that upregulation of immune-associated gene sets after chemotherapy are associated with improved RFS and OS in TNBC. FIG. 6A: Gene set scores were calculated by summing expression levels of all gene set member genes across each candidate gene set (n=100). Changes pre- to post-NAC was then calculated for each TNBC patient (n=44) and each gene set score was tested iteratively in a univariate cox-proportional hazards model for association with RFS (left) or OS (right) after chemotherapy and surgery. Individual gene sets are colored for their statistical significance (red: nominal p-value<0.05; green: q-value (FDR)<0.10; black: not significant). Selected top gene sets are labeled but are limited for clarity. Gene sets with negative coefficients are associated with better outcome, while gene sets with positive coefficients are associated with worse outcome. FIG. 6B: Representative Kaplan-Meier plots for selected gene set changes with beneficial associations are shown (left: T cell activation; right: NK cell functions). Strata are defined by tertiles, and generally represent upregulation during NAC (blue), no change/equivocal (green), and downregulated (red). P-values represent the log-rank test for trend.

FIG. 7A-7E includes data providing evidence of enhanced T cell functionality in the CD8+PD-1HI peripheral compartment. FIG. 7A: Clinical details of 4 patients analyzed prospectively for changes in peripheral blood T cell functionality. NST indicates no special type. FIG. 7B: Polyfunctionality of PD-1HICD4+ and PD-1HICD8+ T cells isolated from PBMCs in 4 patients prior and after NAC (>1000 individual cells/sample/timepoint) was determined by Isoplexis single-cell cytokine profiling. Polyfunctionality is defined as the percentage of cells capable of producing z 2 cytokines following CD3/CD28 stimulation. The percentage of cells in each sample capable of secreting 2, 3, 4, or 5+ cytokines are depicted in stacked bars. Characteristics of each of the 4 patients are shown above the bars. Patients with TNBC (Pt. 1 and Pt. 4) had greater increases in polyfunctionality in the CD8+ compartment with NAC. FIG. 7C: Heatmap representation of log cytokine signal intensity of each cell in each patient sample, pre and post NAC. Each row represents one PD-1HiCD8+ T cell. White indicates no cytokine secreted. FIG. 7D: TCRI3 chain repertoire analysis in CD8+ peripheral blood T cells. Upper plots indicate the number of individual T cells sequenced plotted by sample on the left Y axis; number of clonotypes (unique CDR3 amino acid sequences) plotted by sample on the right Y axis. In the lower graph, each sample is divided into the number of clonotypes comprising expanded (hyper-expanded, large, medium, small, and rare) compositions of the detected repertoire (categories divided by orders of magnitude of fraction of total repertoire). FIG. 7E: The fraction of repertoire clonotypes identified in PD-1HI versus PD-1NEG CD8+ T cells (before or after NAC) classified as ‘hyperexpanded’ or ‘large’ (comprising >0.1% of repertoire). P value represents a 2-sample 2-tailed t-test.

FIG. 8A-8B includes data illustrating changes in immunologic signatures in response to NAC are not associated with outcome in non-TNBC tumors with residual disease. FIGS. 8A and 8B: Volcano plot of the association of changes in immune gene sets (n=100) or immune signatures with RFS or OS. No gene sets were significantly associated with RFS or OS after correcting for multiple comparisons (q<0.10).

FIG. 9A-9D includes data illustrating that changes in sTILs after NAC do not correspond to an observed change in T cell clonality. FIG. 9A: 15 pre- and post-NAC samples (n=30 total) were analyzed by TCRI3 chain sequencing (Adaptive ImmunoSEQ). The imputed number of T cells sequenced in each sample correlates strongly to the number of sTILs analyzed by H&E on adjacent sections. FIG. 9B: Changes in intra-tumor T cell clonality before and after NAC is plotted by individual patient. FIG. 9C: Change in intra-tumor T cell clonality before and after NAC (Post-Pre) is plotted according to TNBC status (TNBC n=8; non-TNBC n=7). Error bars represent mean±sem. P-value represents result of two-sample t-test. FIG. 9D) No correlation was observed between change in sTILs and change in productive TCRI3 clonality across individual patients.

FIG. 10A-10D include data showing that single-cell RNA sequencing of CD8+PD-1HI peripheral T cells from 2 patients with TNBC after NAC demonstrate high expression of cytolytic markers and MHC-II transcripts. FIG. 10A: UMAP plots of 1,964 PD-1HICD8+ peripheral T cells across 2 patients (672 and 1,292 respectively) are shown. Five (5) clusters (0-4) were defined. FIG. 10B: Percent of cells sequenced comprising each cluster are plotted. FIG. 10C: Heatmap identifying abundant transcripts across clusters. A selection of genes defining cluster 0 are highlighted. Data depicted include combined cells from both Pt.1 and Pt.4. FIG. 10D: Cluster 0 was selectively higher for cytolytic transcripts GZMB, GNLY and FGFBP2 (Ksp37).

FIG. 11 includes exemplary results of FACS gating to identify PD-1HI CD8+ T cells from peripheral blood. Cells were gated on singlet-lymphocytes, viability-stain negative, CD3+, CD8+, and the top 20% of PD-1 expressing cells were selected for downstream analysis.

FIG. 12A-12C include data illustrating that an 8-gene activated T cell signature derived from whole blood at surgery is associated with pCR and prognosticates recurrence in RD patients. Individual gene plots of 8 analyzed genes by nanoString from RNA derived from whole blood sampled within 14 days leading up to definitive surgery. Datapoints are stratified by untreated patients (No NAC), those experiencing pCR (pCR), those with RD that did not recur (RD not recur) and those with RD that recurred (RD recur) within 3 years after surgery. Box plots represent the interquartile range. P values represent Kruskal-Wallis tests. * indicates p<0.05 by post-hoc Dunn test. FIG. 12B) A composite gene signature derived as PDCD1+NKG7+LAG3+GZMH+GZMB+GNLY+FGFBP2−HLA-G, stratified by outcome, as in FIG. 12A.

FIG. 12C includes a heatmap showing row-standardized (Z score) gene expression for genes assayed across all patients.

FIG. 13A-13C include data showing that cytokine secretion is markedly enriched in PD-1HI T cells after NAC in a patient with pCR to NAC in TNBC. Cytokines assayed (FIG. 13A) and functional grouping (FIG. 13B) Polyfunctionality of PD-1HI CD4+, PD-1NEG CD4+, PD-1HICD8+ and PD-1NEG CD8+ T cells isolated from PBMCs in Pt. 4 (TNBC; pCR) prior to and after NAC was determined by Isoplexis single-cell cytokine profiling. Polyfunctionality is defined as the percentage of cells capable of producing z 2 cytokines following CD3/CD28 stimulation. The percentage of cells in each sample capable of secreting 2, 3, 4, or 5+ cytokines are depicting in stacked bars. Greater increases in polyfunctionality in the CD8+ compartment with NAC were observed in PD-1HI cells, consistent with the observation that the PD-1HI CD8+ peripheral T cell compartment is enriched for tumor-specific T cellss. FIG. 13C includes a heatmap representation of log cytokine signal intensity of each cell in each patient sample, pre and post NAC. Each row represents one PD-1HiCD4+ T cell. White indicates no cytokine secreted.

FIG. 14 includes a series of graphs illustrating that TCR/3 repertoires in the post-NAC residual disease or tumor scar are most like PD-1^{HI}CD8+ peripheral repertoires. For each patient analyzed, the Jaccard index, normalized for individual sample detected TCR repertoire size, is plotted against the TCRű repertoire detected in the post-NAC tumor (Pts. 1-3) or post-NAC tumor scar (Pt. 4).

FIG. 15 includes the results of a purity-of-sort analysis. UMAP cluster heatmap analysis of expected RNA markers CD4 (not expressed), CD8A (universally expressed), and PDCD1 (universally but variably expressed).

FIG. 16A-16B include results of differential analysis to identify candidate genes for blood-based detection. FIG. 16A: Volcano plot for genes enriched in ‘cluster 0’ versus all others FIG. 16B: Volcano plot for genes enriched in patient 1 versus patient 4. Genes are colored by significance (grey: not significant; green: log 2 fold change>|0.5|; blue: adjusted p<0.05; red: adjusted p<0.05 and log 2 fold change>|0.5|).

FIG. 17A-17C include information and results from the examination of peripheral blood in breast cancer patients. FIG. 17A: Schematic overview of the study. FIG. 17B: Results of gene set enrichment analysis (GSEA) in patients with pathological complete response (pCR) vs. residual disease (RD). FIG. 17C: Enrichment plots showing upregulation of three significant gene sets in patients with pCR.

FIG. 18 includes a heatmap showing row-standardized (Z score) gene expression for genes most strongly enriched in the pCR patients vs the RD patients.

FIG. 19A-19C include complement/IFN signature results for refining cytotoxicity score. FIG. 19A: Scores for the 8 gene cytotoxic signature and the IFN/complement score are shown. FIG. 19B: Composite score of IFN/Complement signature minus cytotoxic signature predicts outcome in breast cancer patients. FIG. 19C: cytotoxic score is primarily expressed in peripheral blood CD8+ T cells and natural killer (NK) cells, while the IFN/Complement score is primarily expressed in monocytes.

FIG. 20A-20E include results of studies looking at cell type. FIG. 20A: CIBERSORTx to deconvolute cell type abundance from bulk gene expression data. FIG. 20B: Monocyte values, as determined by CIBERSORTx, are higher in patients with a pCR compared to those with residual disease or those who did not receive NAC. FIGS. 20C and 20D: Clinically measured relative monocyte values for patients. FIG. 20E: Use of the synthetic derivative, a de-identified medical record system, to identify additional breast cancer patients treated with NAC.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods detecting expression of a combination of genes in a sample from a subject having breast cancer and who has received neoadjuvant chemotherapy (NAC), methods of determining likelihood of residual disease (RD) or pathological complete response (pCR), and methods of providing recommendations for further treatment or cessation of treatment could. The presently-disclosed subject matter provide methods, whereby subjects who could benefit from surgery and/or further treatment could be identified, and subjects identified as having a pCR could ultimately be spared unproductive surgery.

The method as disclosed herein is envisioned for use in connection with a subjecting having breast cancer. In some embodiments, the subject has triple-negative breast cancer (TNBC). The method as disclosed herein is also envisioned for use in connection with a subject who has received neoadjuvant chemotherapy (NAC). In some embodiments, it is possible that the subject will be consider surgery or other additional treatment.

In some embodiments of the presently-disclosed subject matter the method involves obtaining or having obtained a biological sample from the subject; and detecting or having detected expression levels in the sample of genes in one or more signatures.

In some embodiments of the presently-disclosed subject matter the biological sample is a peripheral blood sample. In some embodiments, the biological sample is a buffy coat fraction of the whole peripheral blood, or purified immune cells from whole peripheral blood. In some embodiments, the biological sample is a sample comprising monocytes. In some embodiments, the biological sample is a tumor sample or a sample obtained from the tumor-immune microenvironment. In some embodiments, the biological sample is from a lymph node.

As noted, methods of the presently-disclosed subject matter involve detecting or having detected expression levels in the sample of genes in one or more signatures. Some embodiments make use of a first signature, sometimes referred to herein as a cytotoxic signature or a residual disease (RD) signature, includes the following eight genes PDCD1, NKG7, LAG3, GZMH, GZMB, GNLY, FGFBP2, and HLA-DRB5. In some embodiments of the method, at least five genes of considered. In some embodiments of the method, six, seven, or all eight of the genes of the first signature are considered.

Some embodiments of the method include a step of calculating a first signature score by adding the expression level of each of the genes selected for detection from PDCD1, NKG7, LAG3, GZMH, GZMB, GNLY, and FGFBP2; and subtracting the expression level of HLA-DRB5, if detected. The expression level can be expressed, for example, transcript count for the gene(s) being detected. In some embodiments, the method further involves identifying the subject as having a likelihood of residual disease (RD) when the first signature score is greater than a standardized control; or identifying the subject as having a likelihood of pathological complete response (pCR) when the first signature score is less than a standardized control. The standardized control can be selected according to methods know to those skilled in the art, for example, by detection of normalization genes.

Some embodiments of the method include a step of identifying the subject as having a likelihood of residual disease (RD) and/or cancer recurrence when there is an elevated level of each of the genes selected for detection from PDCD1, NKG7, LAG3, GZMH, GZMB, GNLY, and FGFBP2, and a reduced level of HLA-DRB5, if detected. Some embodiments of the method include a step of identifying the subject as having a likelihood of pathological complete response (pCR) when there is a reduced level of each of the genes selected for detection from PDCD1, NKG7, LAG3, GZMH, GZMB, GNLY, and FGFBP2, and an elevated level of HLA-DRB5, if detected.

Some embodiments of the method further include administering or recommending administration of additional chemotherapy prior to surgery and/or administration of additional chemotherapy after surgery when the subject is identified as having a likelihood of RD; or proceeding or recommending proceeding with surgery without administering additional chemotherapy when the subject is identified has having a likelihood of pCR.

Some embodiments of the presently-disclosed subject matter involve a second signature, sometimes referred to herein as a IFN/complement signature or pathological complete response (pCR) signature. The second signature includes the following sixty genes: SERPING1, IFIT3, IFI44L, IFI44, LAP3, FCGR1A, EPSTI1, IFIT2, TNFSF10, WARS1, IFITM3, MX1, MT2A, BATF2, IL15, IFIT1, STAT1, GBP4, ISG15, OAS3, JAK2, VAMP5, FGL2, PLSCR1, OASL, SAMD9L, USP18, SECTM1, APOL6, PLA2G4A, UBE2L6, CFB, PSME2, OAS2, STAT2, PARP14, CASP1, IFI35, HLA-DMA, GCH1, CD86, IL15RA, DDX60, LATS2, BST2, NMI, IFIH1, CASP4, EIF2AK2, PARP9, GBP2, TENT5A, OAS1, C1QC, C1QA, C2, KYNU, MMP14, PDP1, and CASP10.

In some embodiments of the method, at least ten genes of the second signature are considered. For example, in some embodiments the following ten genes of the second signature are considered: C1QC, CASP10, JAK2, IL15, TNFSF10, C1QA, IFIT3, EPSTI1, PSME2, and LAP3. In some embodiments of the method, more than 10 or even all sixty of the genes of the second signature are considered.

Some embodiments of the method include a step of calculating a second signature score by adding the expression level of each of the genes selected for detection. The expression level can be expressed, for example, transcript count for the gene(s) being detected. In some embodiments, the method also involves identifying the subject as having a likelihood of residual disease (RD) when the second signature score is less than a standardized control; or identifying the subject as having a likelihood of pathological complete response (pCR) when the second signature score is greater than a standardized control.

In some embodiments, the method includes identifying the subject as having a likelihood of residual disease (RD) and/or cancer recurrence when there is a reduced level of each of the genes in the second signature that are selected for detection. In some embodiments, the method includes identifying the subject as having a likelihood of pathological complete response (pCR) when there is an elevated level of each of the genes in the second signature that are selected for detection.

Some embodiments of the method further include administering or recommending administration of additional chemotherapy prior to surgery and/or administration of additional chemotherapy after surgery when the subject is identified as having a likelihood of RD; or proceeding or recommending proceeding with surgery without administering additional chemotherapy when the subject is identified has having a likelihood of pCR.

As will be apparent to the skilled artisan upon studying this document, it some embodiments, a method could make use of both the first signature and the second signature, while in other methods only one of the signatures is employed.

In some embodiment of the presently disclosed subject matter, the method involves extracting mRNA from the biological sample. In this regard, the method can also involve measuring in the extracted mRNA the levels of mRNA of genes from the first signature or genes from the second signature. In some embodiments, method also involves measuring in the extracted mRNA the levels of mRNA of normalization genes to control for the individual sample mRNA content. For example, PTPRC, RPL13a, and/or TBP can be used as normalization genes. As will be recognized by the skilled artisan, measurements of mRNA levels can be made using various technologies known in the art, such as, for example, nanoString mRNA profiling, RNA sequencing, or realtime qPCR.

In some embodiments of the presently-disclosed subject matter, detecting expression levels of genes in a sample can be achieved by using a probe for detecting a gene expression product. In this regard, reference is made to Table 1.

In some embodiments of the presently-disclosed subject matter, expression levels of PDCD1, NKG7, LAG3, GZMH, GZMB, GNLY, FGFBP2, and/or HLA-DRB5 in the sample can be measured using a probe that selectively detects sequence selected from: SEQ ID NO: 1, 4, 7, 10, 13, 16, 19, and 22.

In some embodiments of the presently-disclosed subject matter, expression levels of PDCD1, NKG7, LAG3, GZMH, GZMB, GNLY, FGFBP2, and/or HLA-DRB5 in the sample can be measured using a probe comprising a sequence selected from the group consisting of SEQ ID NO: 2, 3, 5, 6, 8, 9, 11, 12, 14, 15, 17, 18, 20, 21, 23, and 24.

TABLE 1
PDCD1

| | | |
|---|---|---|
| Target | CTTCCCCGAGGACCGCAGCCAGCCCGGCCAGGACTGCCGCTTCCGTGTCA | SEQ ID NO: 1 |
| | CACAACTGCCCAACGGGCGTGACTTCCACATGAGCGTGGTCAGGGCCCGG | |
| Example | TGACACGGAAGCGGCAGTCCTGGCCGGGCTGGCTGCCTGGAGTTTATGTA | SEQ ID NO: 2 |
| Probe A | TTGCCAACGAGTTTGTCTTT | |
| Example | CGAAAGCCATGACCTCCGATCACTCCTGACCACGCTCATGTGGAAGTCACG | SEQ ID NO: 3 |
| Probe B | CCCGTTGGGCAGTTGTG | |

NKG7

| | | |
|---|---|---|
| Target | CTGTGGCGGTCCCCGTCCTGGCTATGAAACCTTGTGAGCAGAAGGCAAGAGC | SEQ ID NO: 4 |
| | GGCAAGATGAGTTTTGAGCGTTGTATTCCAAAGGCCTCATCTGGAGCC | |
| Example | TCTTGCCTTCTGCTCACAAGGTTTCATAGCCAGGACGGGGACCGCGAACCTAA | SEQ ID NO: 5 |
| Probe A | CTCCTCGCTACATTCCTATTGTTTTC | |
| Example | CGAAAGCCATGACCTCCGATCACTCGGCTCCAGATGAGGCCTTTGGAATACAA | SEQ ID NO: 6 |
| Probe B | CGCTCAAAACTCATCTTGCCGC | |

LAG3

| | | |
|---|---|---|
| Target | CTTTTGGTGACTGGAGCCTTTGGCTTTCACCTTTGGAGAAGACAGTGGCGACC | SEQ ID NO: 7 |
| | AAGACGATTTTCTGCCTTAGAGCAAGGGATTCACCCTCCGCAGGCTC | |
| Example | CGCCACTGTCTTCTCCAAAGGTGAAAGCCAAAGGCTCCAGTCACCAAAAGCAG | SEQ ID NO: 8 |
| Probe A | ATAAGGTTGTTATTGTGGAGGATGTTACTACA | |
| Example | CGAAAGCCATGACCTCCGATCACTCCCTGCGGAGGGTGAATCCCTTGCTCTAA | SEQ ID NO: 9 |
| Probe B | GGCAGAAAATCGTCTTGGT | |

GZMH

| | | |
|---|---|---|
| Target | AAAAAAGGGACACCTCCAGGAGTCTACATCAAGGTCTCACACTTCCTGCCCTGGA | SEQ ID NO: 10 |
| | TAAAGAGAACAATGAAGCGCCTCTAACAGCAGGCATGAGACTAAC | |
| Example | GGCAGGAAGTGTGAGACCTTGATGTAGACTCCTGGAGGTGTCCCTTTTTTCCAA | SEQ ID NO: 11 |
| Probe A | TTTGGTTTTACTCCCCTCGATTATGCGGAGT | |
| Example | CGAAAGCCATGACCTCCGATCACTCGTTAGTCTCATGCCTGCTGTTAGAGGCG | SEQ ID NO: 12 |
| Probe B | CTTCATTGTTCTCTTTATCCAG | |

GZMB

| | | |
|---|---|---|
| Target | ACACTACAAGAGGTGAAGATGACAGTGCAGGAAGATCGAAAGTGCGAATCTGACTT | SEQ ID NO: 13 |
| | ACGCCATTATTACGACAGTACCATTGAGTTGTGCGTGGGGGACC | |
| Example | GATTCGCACTTTCGATCTTCCTGCACTGTCATCTTCACCTCTTGTAGTGTCACAATTCT | SEQ ID NO: 14 |
| Probe A | GCGGGTTAGCAGGAAGGTTAGGGAAC | |
| Example | CGAAAGCCATGACCTCCGATCACTCGGTCCCCCACGCACAACTCAATGGTACTGTCGT | SEQ ID NO: 15 |
| Probe B | AATAATGGCGTAAGTCA | |

GNLY

| | | |
|---|---|---|
| Target | TGCCGGCTCCTCGCTTCCTCGATCCAGAATCCACTCTCCAGTCTCCCTCCCCTGACTCCCT | SEQ ID NO: 16 |
| | CTGCTGTCCTCCCCTCTCACGAGAATAAAGTGTCAAGCA | |
| Example | GGAGGGAGACTGGAGAGTGGATTCTGGATCGAGGAAGCGAGGAGCATCCTCTTCTTTT | SEQ ID NO: 17 |
| Probe A | CTTGGTGTTGAGAAGATGCTC | |
| Example | CGAAAGCCATGACCTCCGATCACTCTGCTTGACACTTTATTCTCGTGAGAGGGGAGGACA | SEQ ID NO: 18 |
| Probe B | GCAGAGGGAGTCAGG | |

FGFBP2

| | | |
|---|---|---|
| Target | CTTTCTGGAGTTTGCAGAGTTCAGCAATATGATAGGGAACAGGTGCTGATGGGCCCAAG | SEQ ID NO: 19 |
| | AGTGACAAGCATACACAACTACTTATTATCTGTAGAAGTTT | |
| Example | ATCAGCACCTGTTCCCTATCATATTGCTGAACTCTGCAAACTCCAGAAAGCCTCAAGACCT | SEQ ID NO: 20 |
| Probe A | AAGCGACAGCGTGACCTTGTTTCA | |
| Example | CGAAAGCCATGACCTCCGATCACTCAAACTTCTACAGATAATAAGTAGTTGTGTATGCTTG | SEQ ID NO: 21 |
| Probe B | TCACTCTTGGGCCC | |

HLA-DRB5

| | | |
|---|---|---|
| Target | GAGTGTCATTTCTTCAACGGGACGGAGCGGGTGCGGTTCCTGCACAGAGACATCTATAACC | SEQ ID NO: 22 |
| | AAGAGGAGGACTTGCGCTTCGACAGCGACGTGGGGGAGT | |
| Example | CGCACCCGCTCCGTCCCGTTGAAGAAATGACACTCCAAAGACGCCTATCTTCCAGTTTGATC | SEQ ID NO: 23 |
| Probe A | GGGAAACT | |
| Example | CGAAAGCCATGACCTCCGATCACTCCCTCCTCTTGGTTATAGATGTCTCTGTGCAGGAAC | SEQ ID NO: 24 |
| Probe B | | |

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Patients

Three cohorts of patients were combined for the tumor profiling study. All included patients received neoadjuvant therapy and had residual disease and matched pre-treatment tissue was required for inclusion. All but 2 (ER+) patients received cytotoxic chemotherapy as part of their regimen. Four patients (ER+) received courses of hormone therapy as part of their neoadjuvant regimen, two of which were in conjunction with cytotoxic chemotherapy. Five patients (HER2+) received HER2-directed therapy as part of their neoadjuvant regimen.

For the ‘Peru’ cohort, clinical characteristics and molecular analysis of the patients (n=48 with matched pre-treatment tissue) were previously described at the Instituto Nacional de Enfermedades Neoplásicas²¹. Clinical and pathologic data were retrieved from medical records under an institutionally approved protocol (INEN IRB 10-018). For the ‘VICC’ cohort, which included PBMC and whole blood analyses, clinical and pathologic data were retrieved from medical records under an institutionally approved protocol (VICC IRB 030747). For the DARTMOUTH cohort patient samples were collected under a protocol approved by the Dartmouth College Institutional Review Board and the waiver of the subject consent process was IRB-approved. (IRB 28888). Metadata for the primary cohort of patients was provided. For the peripheral blood study, all blood was collected within 14 days preceding definitive surgery. Metadata for the cohort of patients used for the peripheral blood study was provided.

### Example 2: Summary of Studies

The expression patterns of immune-related genes was examined before and after NAC in a series of 83 breast tumors, including 44 TNBCs, from patients with RD. Changes in gene expression patterns in the TIME were tested for association with recurrence-free (RFS) and overall survival (OS). T cell receptor sequencing (TCRseq) was performed on a subset (n=15) of tumors. Additionally, in four patients undergoing NAC, PD-1-high and PD-1-negative CD3+CD8+ peripheral blood mononuclear cells (PBMCs) were profiled using single-cell RNA sequencing (scRNAseq) and multiplexed cytokine secretion assays. Finally, a scRNAseq-derived signature of activated cytolytic cells was used to measure immune activation in the peripheral blood of 36 patients after NAC (collected within 2 weeks prior to surgery) and 24 untreated patients. The association of this signature was tested with pCR and post-surgical cancer recurrence.

### Example 3: Tumor-Infiltrating Lymphocytes Quantification

Stromal tumor-infiltrating lymphocytes were analyzed using full face H&E sections from pre-NAC diagnostic biopsies or post-NAC RD surgical specimens. Samples were scored according to the International TILs Working Group Guidelines²²⁻²⁴. The pre-defined cut point of 30%⁴ was used for all survival analyses.

### Example 4: NanoString nCounter Analysis

Gene expression and gene set analysis on pre- and post-NAC formalin-fixed tissues were performed using the nanoString Pan-Cancer Immunology panel (770 genes) according to the manufacturers' standard protocol. Data were normalized according to positive and negative spike-in controls, then endogenous housekeeper controls, and transcript counts were log transformed for downstream analyses. Normalized linear data was obtained. Gene sets were calculated by summing the log 2-transformed normalized NanoString counts for all genes contained in a given gene set. Samples were simultaneously assayed for PAM50 molecular subtyping.

Briefly, 10 μm sections of diagnostic biopsies or residual tumors were used for RNA preparation (Promega Maxwell 16 RNA FFPE) and 50 ng of total RNA>300 nt (assayed on a Agilent Tapestation 2200 Bioanalyzer) was used for input into nCounter hybridizations for Pan-Cancer Immunology panels or 500-1000 ng RNA for PAM50 analysis. Data were normalized according to positive and negative spike-in controls, then endogenous housekeeper controls, and transcript counts were log transformed for downstream analyses. Subtype prediction was performed in R using the genefu package.

For the 8 gene signature analysis in whole blood, a custom NanoString Elements was constructed to measure the gene expression levels of PDCD1, NKG7, LAG3, GZMH, GZMB, GNLY, FGFBP2, HLA-DRB5, and HLA-G, as well as 3 normalization control genes (PTPRC, RPL13a, TBP). RNA was isolated from whole blood (Promega Maxwell 16 Simply RNA Blood) and 150-250 ng was used for input into the nCounter analysis. Data were normalized as above. Linear normalized data were obtained.

### Example 5: Isoplexis (Single-Cell Cytokine Profiling)

On day 1, cryopreserved PBMCs were thawed and resuspended in complete RPMI media with IL-2 (10 ng/ml) at a density of 1-5×10⁶ cells/ml. Cells were recovered at 37° C., 5% CO2, overnight. Plates were prepared by coating with antihuman CD3 (10 μg/ml in PBS, 200-300 μl/well) in a 96-well flat-bottom plate at 4° C., 0/N. On day 2, non-adherent cells for each sample were collected and viability was confirmed, with dead cell depletion by Ficoll.

For each sample, where sufficient, volume was split in half for each of the following negative isolations: with one half of cells from each sample, CD4 T cells were isolated with CD4+ negative isolation kit following Miltenyi protocol (130-096-533); with the other half of cells from each sample, CD8 T cells were isolated with CD8+ negative isolation kit following Miltenyi protocol (130-096-495). The PD-1+ and PD-1−subsets were from isolated CD4 or CD8 T cells by staining with PE-conjugated anti-PD-1 antibody using the manufacturer's protocol (Miltenyi, 130-096-164) as follows: 1) stain each subset with 10 ul stain:100 ul Robosep buffer for every 1×10\hat{ }7 total cells; 2) incubate at 4° C. for 10 mins; 3) rinse cells by adding 1-2 mL of Robosep and C/F at 300×g for 10 mins; 4) aspirate supernatant and resuspend cells pellets in 80 ul buffer per 1×10⁷ total cells. PD-1+ cells were then isolated with anti-PE microbeads following the manufacturer's protocol (Miltenyi, 130-097-054).

Cells were resuspended in complete RPMI media at a density of 1×10⁶/ml and seeded into wells of the CD3-coated 96-well flat-bottom plate with soluble anti-human CD28 (5 ug/ml). Plates were incubated at 37° C., 5% CO2 for 24 hrs. On day 3, supernatants (100 ul per well) were collected from all wells and stored at −80° C. for population assays. T cells were collected and stained with Brilliant Violet cell membrane stain and AlexaFluor-647-conjugated anti-CD8 at RT for 20 min. Cells were resuspended in complete RPMI media for single-cell Isoplexis assay (human T cell panel), performed according to the manufacturer's standard protocol. Data were collected and analyzed 24 hours later (day 4).

### Example 6: TP53 Sequencing

TP53 gene sequencing was performed using either the Foundation Medicine assay as previously reported²¹ or using the SureMASTR TP53 sequencing assay (Agilent). For the later, purified DNA from FFPE breast tumor sections were amplified and sequenced according to the manufacturer's standard protocol. Samples were sequenced to a depth of −10,000 and mutations were called using the SureCall software (Agilent). Mutation allele frequency was set at 5% and only likely functional (early stops, frameshift deletions and known recurrent hotspot single-nucleotide variation mutations) were selected for sample annotation.

### Example 7: T Cell Receptor Sequencing

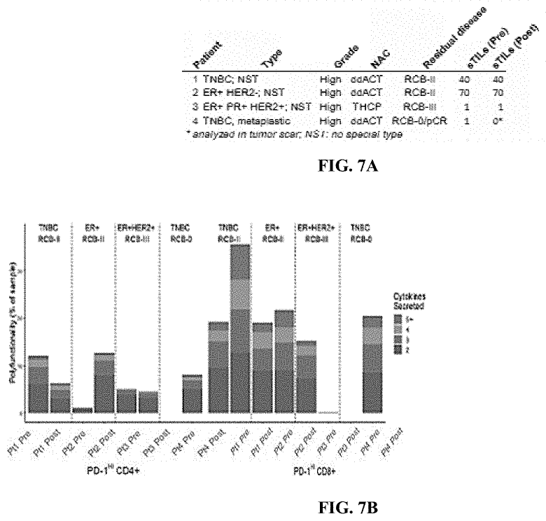

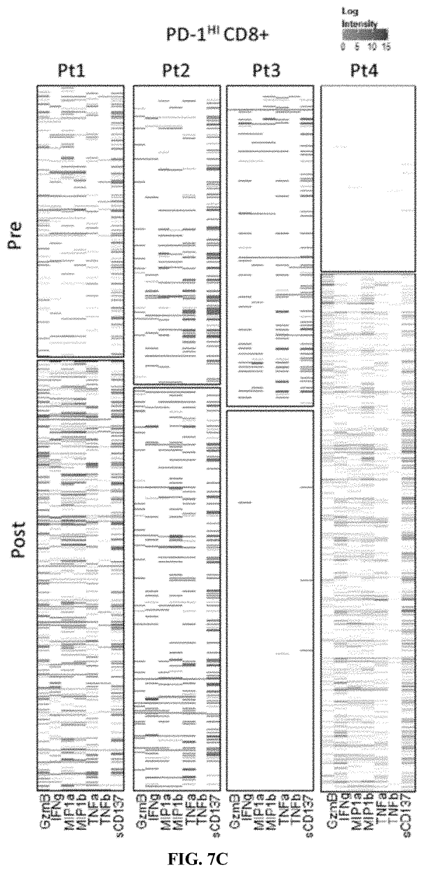

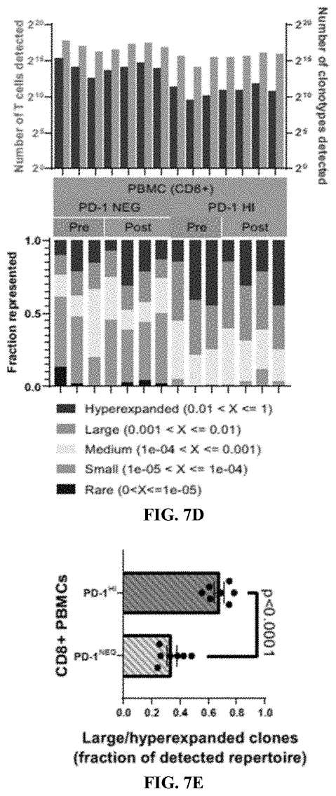

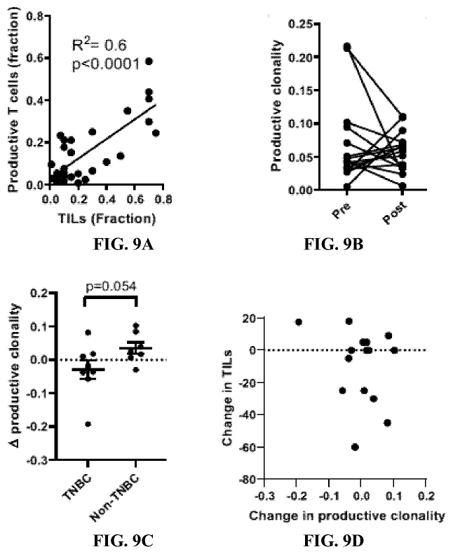

TCR sequencing and clonality quantification was assessed in FFPE samples of breast cancer specimens or PBMCs. For FFPE tissue, DNA or RNA was extracted from 10 μm sections using the Promega Maxwell 16 FFPE DNA or FFPE RNA kits and the manufacturer's protocol. For PBMCs, PD-1^{HI} and PD-1^{NEG} CD8+ T cells sorted by fluorescence-activated cell sorting from samples isolated from EDTA collection tubes and processed using a Ficoll gradient. At least 100K cells were collected, centrifuged, and utilized for RNA purification. TCRs were sequenced using survey level immunoSEQ™ (DNA; Adaptive Biotechnologies) and the Immunoverse assay (RNA; ArcherDX), as previously described^{39,40}. Sequencing results were evaluated using the immunoSEQ analyzer version 3.0 or Archer Immunoverse analyzer. CDR3 sequences and frequency tables were extracted from the manufacturers' analysis platforms and imported into R for analysis using the Immunarch package (immunarch.com)²⁵ in R. Shannon entropy, a measure of sample diversity, was calculated on the clonal abundance of all productive TCR sequences in the data set. Shannon entropy was normalized by dividing Shannon entropy by the logarithm of the number of unique productive TCR sequences. This normalized entropy value was then inverted (1—normalized entropy) to produce the ‘clonality’ metric. TCR6 clonotypes and metadata based on the primary cohort (FIG. 9; Adaptive) were obtained. TCR6 clonotypes and metadata based on prospectively-collected peripheral blood and tumor from FIG. 7 (Archer) were obtained.

### Example 8: Single-Cell RNA Sequencing

PD-1^{HI} and PD-1^{NEG} CD8+ T cells were sorted by fluorescence-activated cell sorting from peripheral blood mononuclear cells isolated from EDTA collection tubes and processed using a Ficoll gradient. Each sample (targeting 5,000 cells/sample) was processed for single cell 5′ RNA sequencing utilizing the 10× Chromium system. Libraries were prepared using P/N 1000006, 1000080, and 1000020 following the manufacturer's protocol. The libraries were sequenced using the NovaSeq 6000 with 150 bp paired end reads. RTA (version 2.4.11; Illumina) was used for base calling and analysis was completed using 10× Genomics Cell Ranger software v2.1.1. Data were analyzed in R using the filtered h5 gene matrices in the Seurat^{26,27} package (R). Briefly, samples were merged, and all cells were scored for mitochondrial gene expression (a marker of dying cells) and cell cycle genes to determine phase. Data were transformed using SCTransform, regressing against mitochondrial gene expression and cell cycle phase. Dimensional reduction was performed using Harmony²⁸.

### Example 9: Stromal Tumor-Infiltrating Lymphocytes (sTILs) in Residual Disease Prognosticate Improved Outcomes in TNBC Patients with Incomplete Response to Neoadjuvant Chemotherapy (NAC)

Immune-related gene expression patterns were examined before and after neoadjuvant chemotherapy (NAC) in a series of 83 breast tumors, including 44 Triple-Negative Breast Cancers (TNBC), from patients with residual disease (RD). Changes in gene expression patterns in the tumor-immune microenvironment (TIME) were tested for association with recurrence-free (RFS) and overall survival (OS). Additionally, the systemic effects of NAC were characterized through single cell analysis (RNAseq and cytokine secretion) of PD-1HI CD8+ peripheral T cells and examination of a cytolytic gene signature in whole blood.

Matched archived pre-treatment (diagnostic biopsy) and post-treatment (residual disease surgical specimen) tumor specimens were procured from the series of 83 patients, including the 44 TNBC patients.

Importantly, the study was refined to include only patients who had residual disease at surgery for analysis, thereby excluding patients who achieved pCR. This was a purposeful selection strategy, as patients with pCR usually experience good outcomes, and the focus was instead on patients with RD for whom additional treatment strategies could eventually replace “watchful waiting” approaches. Metadata for the patients, including treating institution, molecular subtype (PAM50), recurrence-free and overall survival (RFS and OS, respectively), TP53 mutation status, and other molecular and clinical data were available.

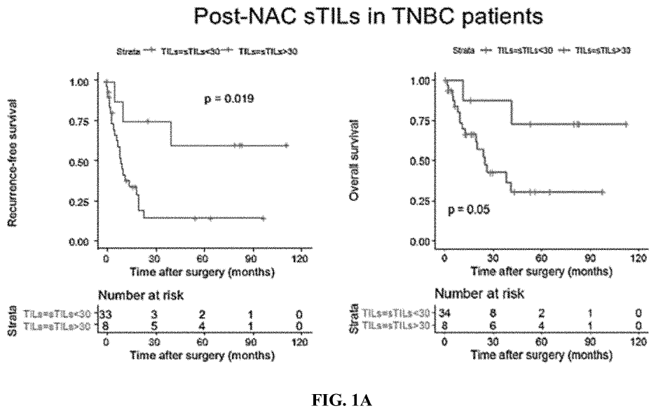

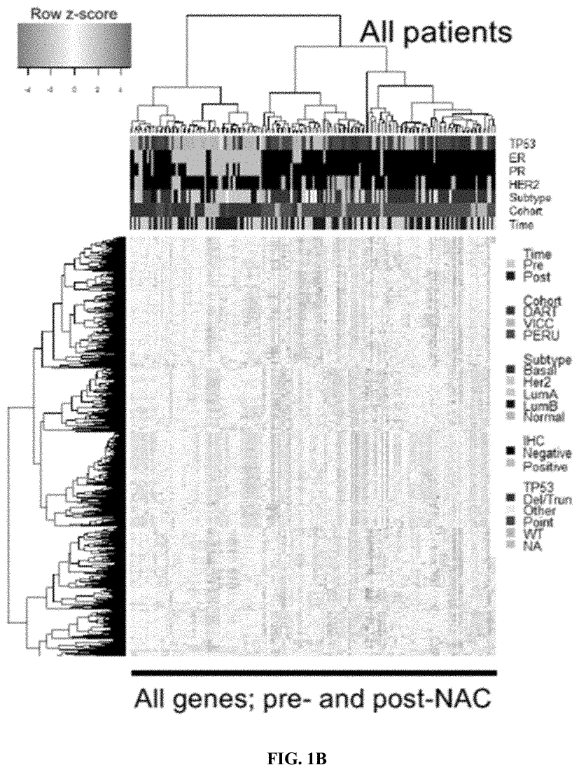

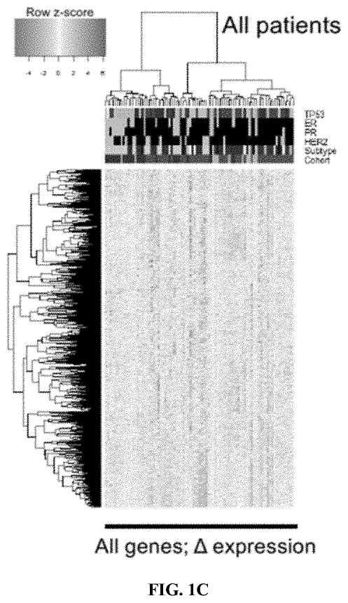

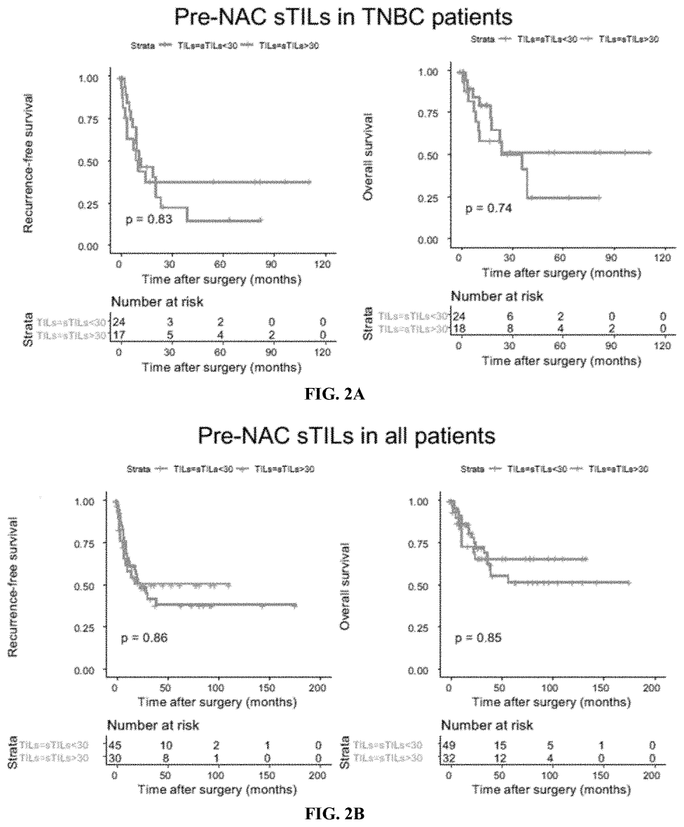

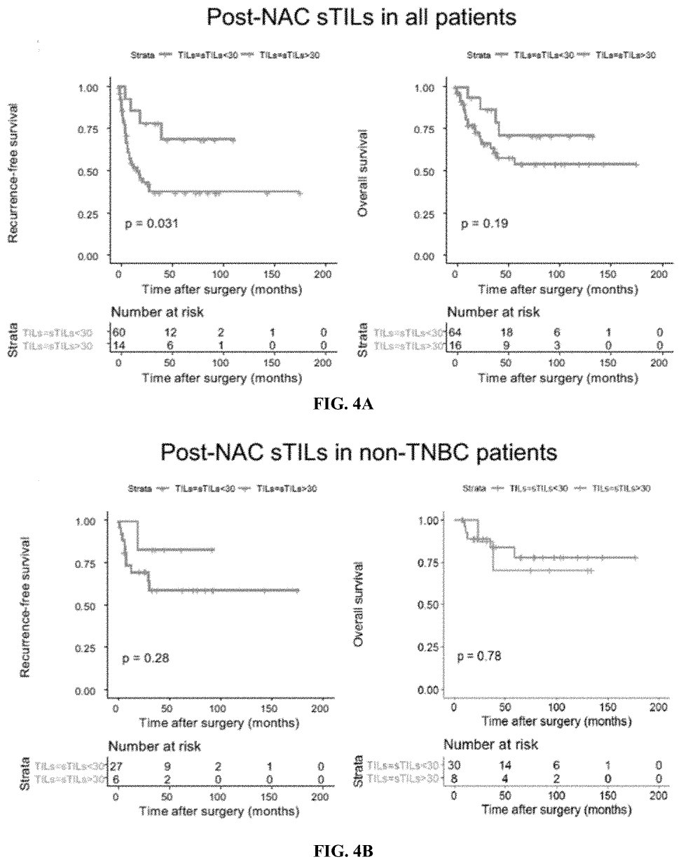

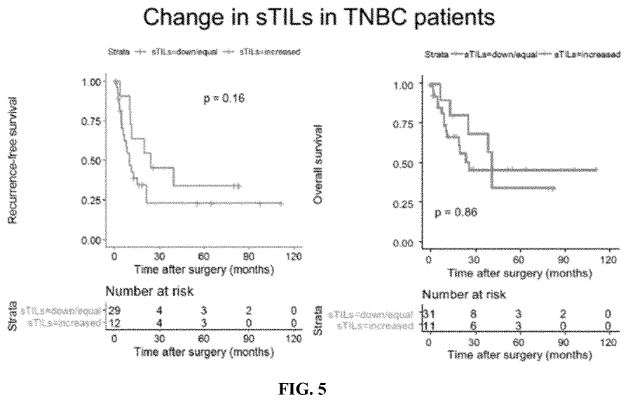

As sTILs have been described and rigorously validated as both a prognostic factor (in surgical specimens for post-surgical outcomes), and a predictive factor (in diagnostic biopsies for benefit from NAC) in TNBC, but not in other breast cancer subtypes, an initial inquiry was whether these findings were consistent with the study cohort. Using the published cutoff (30%) of sTILs⁴, higher abundance of sTILs in the post-NAC residual disease in TNBC patients (n=44) was found to be significantly prognostic for both RFS (log-rank p=0.019) and OS (p=0.05; FIG. 1A). Interestingly, pre-NAC sTILs in the diagnostic biopsy were not prognostic for outcomes in TNBC patients (FIG. 2A), presumably due to the selection strategy of including only patients who lacked pCR. Consistent with prior literature that the prognostic and predictive effect of sTILs is confined to TNBC, neither pre-NAC nor post-NAC sTILs were prognostic for OS when considering the entire cohort (n=83). However, post-NAC sTILs were prognostic for RFS (p=0.031) in the whole cohort (FIG. 2B, 3A). This effect seems primarily driven by TNBC tumors as post-NAC sTILs are not prognostic of either RFS or OS in non-TNBCs (FIG. 4B). Stratifying TNBC patients by whether sTILs were qualitatively increased or decreased/equivocal in the surgical resection compared to the diagnostic biopsy did not provide any prognostic capability in this cohort (FIG. 5).

Thus, in the cohort, abundance of sTILs has the strongest prognostic effect for the post-NAC surgical resection specimen in TNBC tumors with an incomplete response to NAC. These findings, consistent with both retrospective studies and analyses from randomized controlled trials, prompted more detailed molecular studies aimed at understanding how NAC influences the TIME.

### Example 10: Suppression of Immunologic Gene Expression with NAC in TNBC

To measure transcriptional changes occurring in the tumor-immune microenvironment (TIME) induced by NAC, gene expression profiling was performed for a series of 770 immune-related genes using nanoString (Pan-Cancer Immune Panel), before and after NAC in the entire cohort (n=83). Transcriptional patterns and hierarchical clustering for all data primarily segregated tumors based on receptor status (ER/PR/HER2) and/or molecular subtype, with most luminal/hormone receptor-positive tumors appearing in the first cluster, most HER2-positive tumors in the second cluster, and most basal-like/TNBC tumors in the third cluster (FIG. 1B). Examining the data as the change in gene expression for each gene after NAC in a patient-matched fashion (A expression; post-NAC minus pre-NAC) yielded similar patterns, with a trend of most TNBC patients having generalized decreased immune gene expression patterns after NAC (FIG. 1C).

### Example 11: NAC-Induced Immunologic Gene Expression is a Positive Predictor of Outcome in TNBC

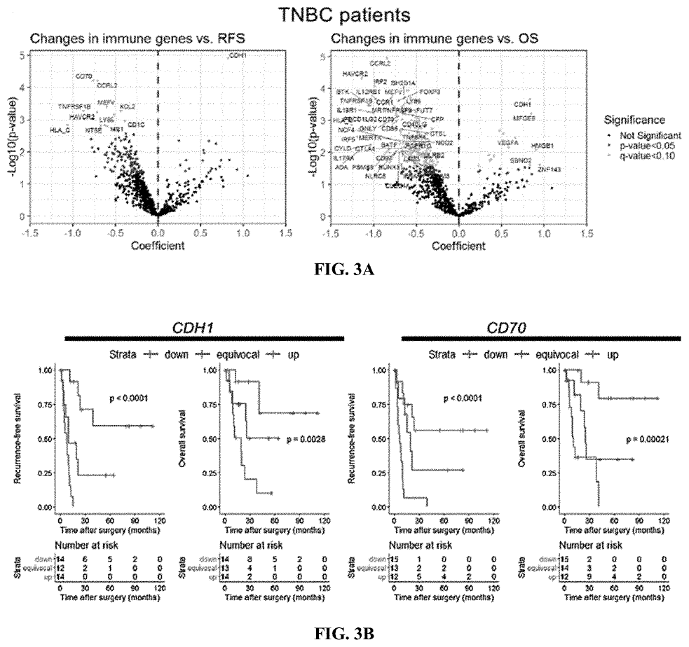

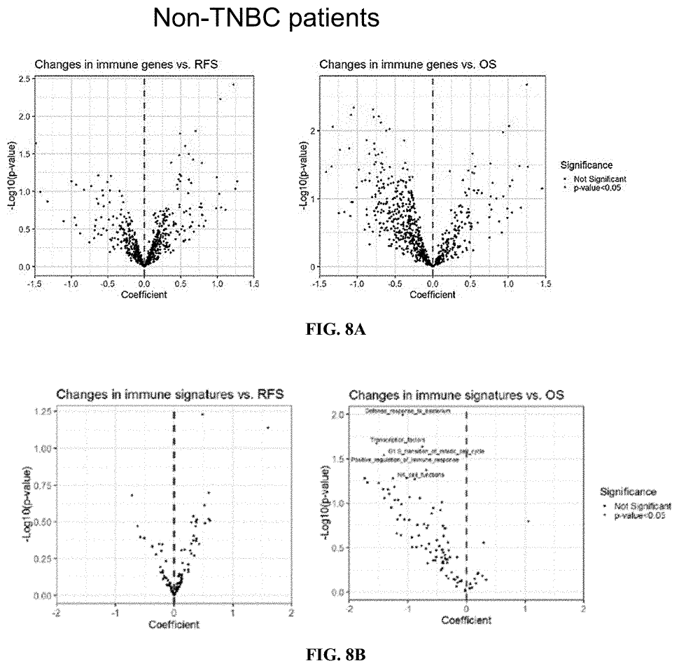

While the TIME change in sTIL abundance did not prognosticate outcome in TNBC patients, changes in individual immune-related genes were examined for association with outcome. Iterative Cox proportional hazards models were performed, using the delta (A) of each gene (post-NAC minus pre-NAC) in an independent univariate analysis, for both RFS and OS. All analyses are reported using a nominal p-value as well as a false discovery rate (FDR; Benjamini-Hochberg method) q-value for association with RFS or OS. After correction for FDR (q<0.10), upregulation of 11 genes were associated with improved RFS, while upregulation of only one gene was significantly associated with worse RFS (CDH1, which encodes e-cadherin) in the TNBC cohort. Interestingly, e-cadherin is known to interact with killer cell lectin-like receptor G1 (KLRG1), an inhibitory receptor expressed by memory T cells and NK cells⁹. In contrast, upregulation of a larger number of genes was associated with improved OS (n=189) or reduced OS (n=15) at FDR q<0.10 (FIG. 3A). Kaplan-Meier visualization examples of strongly prognostic genes (negative prognostic: CDH1; positive prognostic: CD70) reinforced the prominent association of TNBC disease outcomes with changes in immune gene expression during NAC (FIG. 3B). Conversely, no changes in immune-related gene expression were significantly associated with RFS or OS in non-TNBC patients at q<0.10 (FIG. 8A).

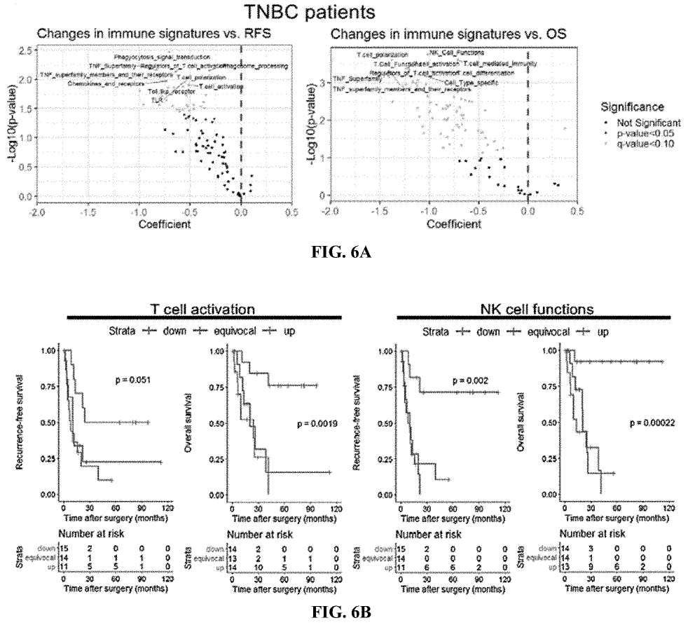

Dimensional reduction through collapsing individual genes into pathways or defined functions can improve interpretation of high-dimensional data. Thus, the gene expression data was collapsed into bioinformatically-categorized immune signatures (sum-scores, defined as the summation of the log 2 expression values for all genes in a category). Organization of the data in this manner and testing the signatures (n=100) for association with RFS and OS yielded a surprising finding—nearly all significant (q<0.10) gene sets (n=37 for RFS and n=77 for OS) identified in this analysis were associated with good outcome (FIG. 6A). Upregulation of only one gene set was significantly associated (q<0.10) with worse OS (“G2 phase and G2/M transition”, which is not an immune-specific gene set). Many of the top-scoring gene sets were associated with T cells, including “T cell polarization”, “T cell immunity”, “T cell activation”, among others. Although manual inspection revealed some overlap in these gene sets, they were largely composed of signature-exclusive genes. Kaplan-Meier visualization examples of strongly prognostic gene sets (“NK cell functions” and “T cell activation”) reinforced the considerable association of changes in immune gene sets during NAC with outcomes (FIG. 6B). Interestingly, and consistent with previous studies on sTILs where little association was observed between immunologic features and outcome, no gene or gene set was significantly associated with RFS or OS in non-TNBC patients at q<0.10 (FIG. 8A-B). Thus, these data suggest that NAC, exclusively in TNBC, could promote immunologic activity leading to improved outcomes in a subset of patients. However, these effects may be related to factors beyond TNBC biology, as hormone-receptor-positive patients receive additional endocrine therapy in the adjuvant setting, complicating associations with RFS and possibly OS. Nonetheless, immune-related signatures, particularly those derived from T cells, appeared to be strongly associated with improved outcomes in TNBC.

### Example 12: Changes in T Cell Clonality and Function in Tumors and Peripheral Blood Induced by NAC

A robust T cell response is characterized by oligoclonal expansion of antigen-specific T cells. Therefore, it was determined whether clonality of T cells in the TIME was altered during NAC. In a subset of samples (n=15; 8 TNBC, 7 non-TNBC), T cell receptor (TCR) 6 chain sequencing was performed using the ImmunoSeq assay to estimate the number of unique T cell clones (diversity), and the presence of expanded T cell clones in the TIME before and after NAC. Given the breadth of sTILs fractions observed among breast tumors as well as caveats associated with comparison of samples derived from diagnostic core needle biopsies vs. surgical resections, it was first verified that the number of productive T cells was associated with estimation of sTILs determined on adjacent sections. A strong association was detected between these parameters (R²=0.6; p<0.0001; FIG. 9A), raising confidence in the assay results. In this sample set, NAC did not universally alter productive clonality (FIG. 9B), a measurement of the number of times the same (productive) TCR6 sequence is represented in the sample, which is a descriptor of T cell clonal expansion. When stratified by breast cancer subtype, there was no significant change in productive clonality with NAC (one-sample t-test). However, TNBC tumors demonstrated a qualitative trend toward decreased clonality after NAC, while non-TNBC tumors trended toward increased clonality after NAC. The difference between these two subgroups approached significance (p=0.054; two-sample t-test; FIG. 9C). There was no association of change in clonality with change in sTILs, suggesting that changes in sTIL abundance after NAC are not necessarily due to expansion of existing clones (FIG. 9D).

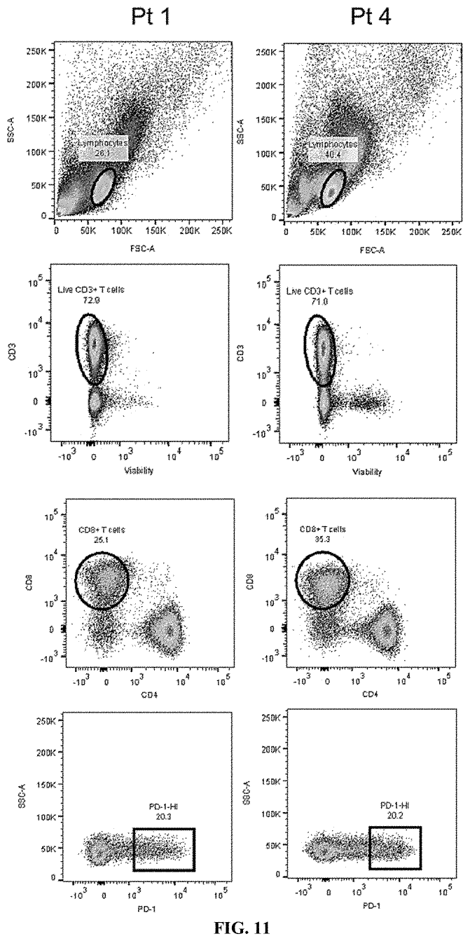

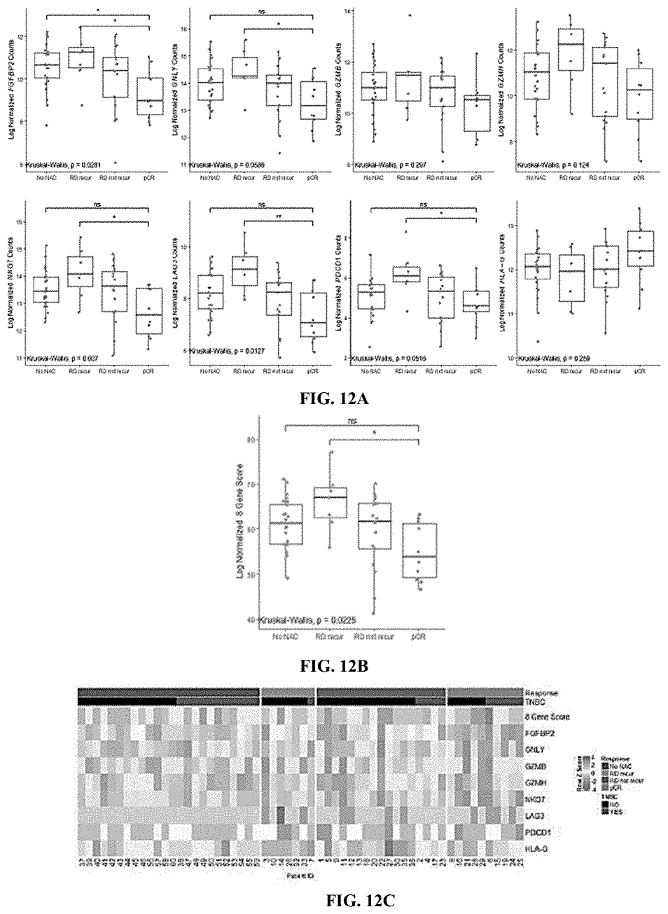

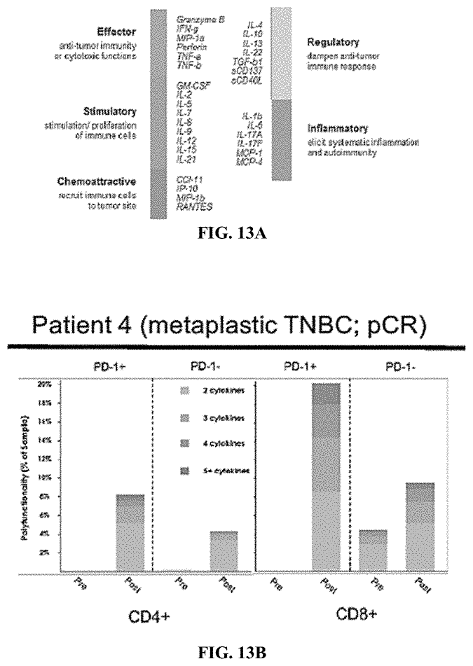

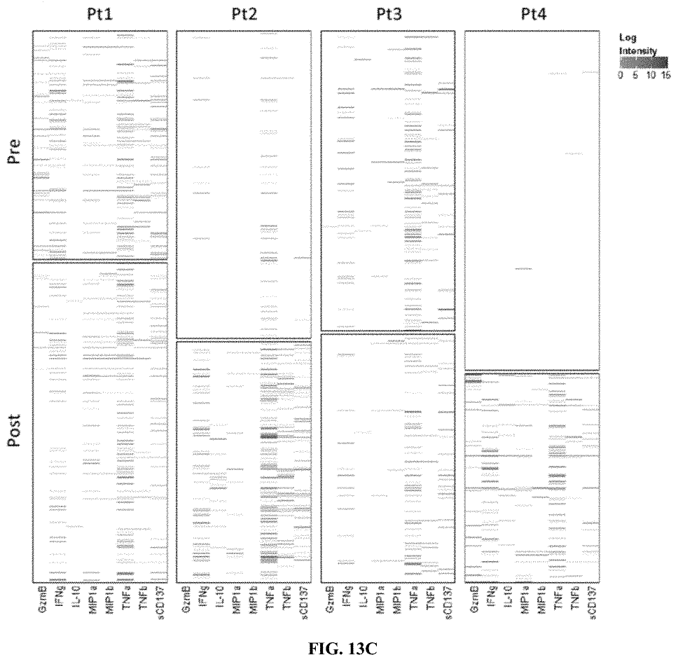

To further explore changes in T cell clonality and function in response to chemotherapy, PBMCs were prospectively collected from four breast cancer patients (including two TNBCs) before and after NAC (FIG. 7A). In addition, the post-NAC residual disease (or in one case, pCR residual scar) was analyzed in tandem. Based on previous findings demonstrating that tumor-reactive T cells are enriched in the CD8+PD-1^{HI} population of peripheral T cells⁸, CD4+ and CD8+ cells were purified from each sample by fluorescence-activated cell sorting (FACS), further stratifying by PD-1-negative (PD-1^{NEG}) and PD-1^{HI} (top 20% expressers of CD8+ or CD4+ cells) status (gating scheme shown in FIG. 11). Using a functional assay of cytokine (32-plex, FIG. 13A) secretion following CD3/CD28 stimulation, PD-1^{HI} peripheral T cells was determined to have functional capacity, secreting multiple cytokines following activation, and these effects were particularly pronounced in CD8+ T cells (FIG. 13B). In 2/2 TNBC patients, the percentage of ‘polyfunctional’ PD-1^{HI}CD8+ T cells—those capable of expressing multiple cytokines after TCR stimulation—were increased following NAC (FIG. 7B). In contrast, 2/2 ER+ breast cancer patients experienced a drop or stasis in the functionality of the PD-1^{HI}CD8+ population of cells following NAC (FIG. 7B). Of note, the patient with ER+HER2+ disease has a near complete loss of T cell functionality after NAC. Cytokines produced by individual PD-1^{HI}CD8+ cells in TNBC patients were primarily effector (e.g., Granzyme B, IFN-y, MIP-1a, TNF-a, and TNF-13) and chemo-attractive (MIP-113) cytokines (FIG. 7C). PD-1^{HI}CD4+ T cells also produced primarily effector cytokines including IFN-γ and TNF-α (FIG. 13C).

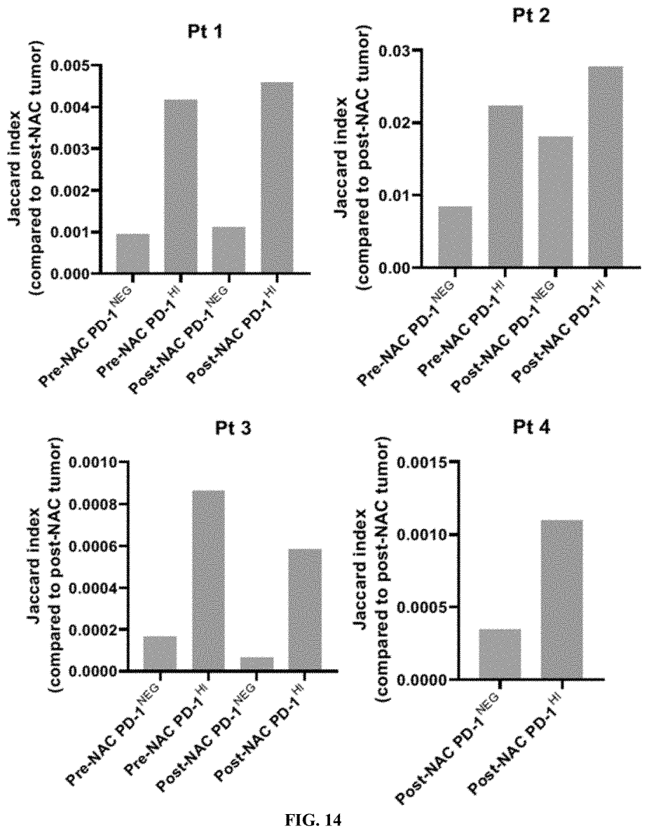

PD-1^{HI}CD8+ and PD-1^{NEG} CD8+ T cells from pre-NAC and post-NAC blood (except patient 4, for whom a sufficient pre-NAC sample was not available) were also analyzed by TCR sequencing. While the number of detected T cells was consistent among all samples, the clonotypes detected (unique TCRs) were considerably lower in PD-1^{HI} CD8+ T cells (FIG. 7D). This suggests that there are more repetitive sequences detected in the PD-1^{HI} population, indicating clonal expansion. Consistent with this observation, the proportion of the overall TCR repertoire occupied by expanded clonotypes (large or hyperexpanded clonotypes consisting of greater than 0.1% or 1% of the total repertoire, respectively) was substantially higher in the PD-1HI than in PD-1^{NEG} CD8+ T cell fractions (FIG. 7E). The TCR repertoire was also sequenced in the post-NAC residual disease, although the number of T cells sequenced in these samples were limited due to fixation of tissue and small T cell abundance as a function of total RNA in the bulk samples, and thus should be interpreted with caution. Nonetheless, the similarity (Jaccard index, normalized to size of repertoire detected) of tumor-infiltrating TCRs in the post-NAC sample was found to be universally more similar to the PD-1^{H1}CD8+ peripheral TCR repertoires, compared to the PD-1^{NEG} CD8+ repertories (FIG. 14). This suggests that the PD-1^{HI} peripheral compartment is enriched for similarity to TILs relative to the PD-1^{NEG} peripheral compartment.

### Example 13: Single-Cell RNAseq of Peripheral PD-1^{HI} CD8+ T Cells Identifies a Unique Population of Cytolytic Effector Cells

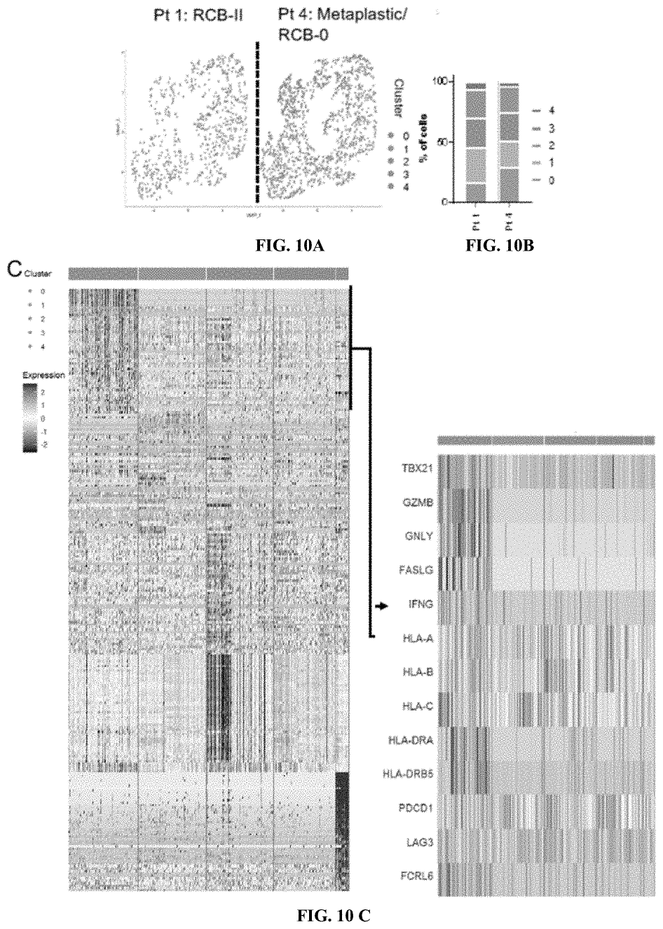

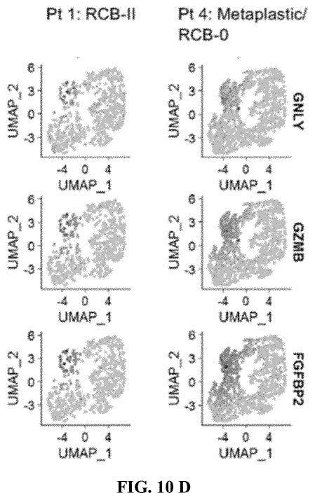

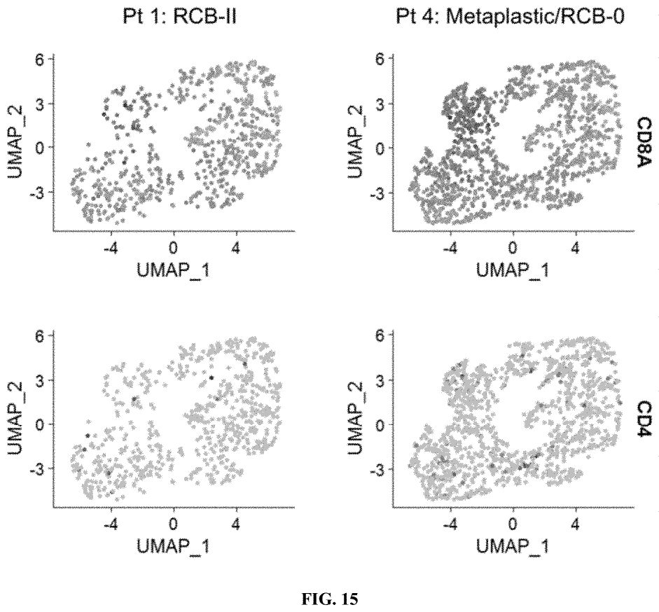

Next, scRNAseq was used to describe the post-NAC peripheral PD-1^{HI}CD8+ T cell populations at the time of surgery in the blood of two TNBC patients: one with residual disease (Pt. 1) and one with matrix-producing metaplastic TNBC who experienced pCR (Pt. 4). Uniform Manifold Approximation and Projection (UMAP) analysis was performed on Harmony-normalized samples to adjust for inter-sample technical variation, and cells were stratified based on 5 clusters identified through the Louvain algorithm. Although the composition of the cells was largely similar, one cluster (cluster 0′) was identified which was enriched in Pt. 4 (FIG. 10A-B). Examination of genes differentially expressed in this cluster of cells suggested a cellular identity concordant with that of highly cytotoxic memory (TBX2/-expressing) T cells, which had an abundance of MHC-I (HLA-A/B/C) and MHC-II (e.g., HLA-DRA, HLA-DRB5) family member expression as well as expression of cytolytic and immune checkpoint genes (e.g., LAG3, FCRL6¹⁰, and higher transcriptional expression of PDCD1; FIG. 10C). Verification of the pattern of expression of key cytolytic and killer-identity genes [GNLY (granulysin), GZMB (granzyme B), and FGFBP2 (killer-secreted protein 37)] showed that these genes were almost exclusively expressed in cluster 0 (FIG. 10D). This analysis also demonstrated purity-of-sort in that all clusters expressed CD8A and PDCD1, but not CD4 (FIG. 15).

These data led to two competing hypotheses: 1) Cluster 0 genes, reflective of cytolytic CD8+ T cells, are a positive prognostic factor reflective of robust anti-tumor immunity as evidenced by their enrichment in the metaplastic TNBC patient with pCR; or 2) Cluster 0 genes are reflective of ongoing disease including the micrometastatic component that cannot be sampled from the primary tumor. The second hypothesis is supported by the observations that pCR is less prognostic of RFS and OS in metaplastic disease^{11,12} and that rates of recurrence following chemotherapy are higher for metaplastic disease than non-metaplastic TNBC^{11,13,14}. Interestingly, matrix-producing metaplastic breast cancer (Pt. 4) has been shown to be associated with pCR to NAC, but often can still recur despite pCR^{11,15}. Follow-up for this individual patient was immature at the time of reporting, and thus recurrence, and therefore presence of micrometastatic disease at the time of sampling, cannot be ruled out.

### Example 14: Cytolytic Gene Expression Signatures are Present in Blood and Associated with Increased Likelihood of Recurrence

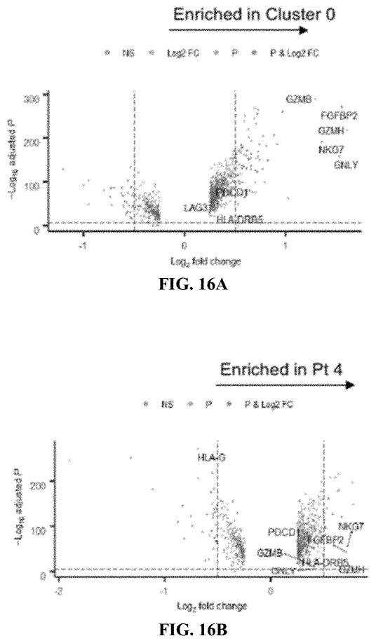

To determine whether cytolytic signatures representative of cluster 0 genes were associated with disease outcome, archived whole blood was evaluated from a series of 60 breast cancer patients. All samples were collected within 14 days preceding surgical resection for primary breast cancer, with 36 samples having received NAC, in addition to 24 samples from untreated patients. A series of eight genes enriched in cluster 0 (PDCD1, NKG7, LAGS, GZMB, GNLY, FGFBP2, HLA-DRB5), one gene enriched in Pt 1 (RCB-II) over Pt 4 (pCR; HLA-G) (FIG. 16), and three normalization control genes (PTPRC, RPL13a, TBP)¹⁶ were selected for a 12-gene custom NanoString gene expression analysis. HLA-G has been described as an immune checkpoint which can dampen anti-tumor immune responses¹⁷⁻¹⁹, and thus HLA-G expression was expected to be inversely correlated with the other selected genes, as is the case in the scRNAseq dataset. One of these genes performed poorly (HLA-DRB5), likely due to frequent polymorphisms in the gene leading to highly variable probe binding and was therefore omitted from further analysis. Information on the presence of pCR/RD at surgery, ER/PR/HER2 status, and clinical follow-up (recurrence at 1000 days after surgery for RD patients) was collected.

Nearly all tested genes demonstrated a pattern supporting the hypothesis that gene expression in whole blood is associated with ongoing disease, being highest (or lowest in the case of HLA-G) in untreated patients (who have ongoing tumor burden by virtue of not having received therapy prior to surgery) and those with RD compared to those with pCR. Furthermore, among patients with RD, higher expression (or lower in the case of HLA-G) tended to be observed in patients who had early recurrences in the first 3 years following surgery (and thus may have had micrometstatic disease at the time of surgery). Several of these genes (FGFBP2, GNLY, PDCD1, LAGS, and NKG7) were also significantly differentially expressed or approached statistical significance across the outcome groups (Kruskal-Wallis test). Comparisons were particularly striking between the group of patients with RD who experienced early disease recurrence and the group with pCR following NAC (post-hoc Dunn test; FIG. 13A). A composite score of PDCD1+NKG7+LAG3+GZMH+GZMB+GNLY+FGFBP2 HLA-G also demonstrated statistically significant associations with presence of ongoing disease (FIG. 13B). Interestingly, expression levels of these genes did not always correlate with one another, indicating heterogeneity in their expression patterns and some degree of independence (FIG. 13C). Trends in gene expression were similar for TNBC and non-TNBC patients, but in-depth subgroup analyses were limited by sample size. Thus, peripheral anti-tumor immunity in blood may be a useful measure of persistent residual primary or micrometastatic disease and could identify patients likely to benefit from additional therapy.

### Discussion Related to Examples 1-14

In these Examples, the prognostic nature of sTILs in the RD of TNBC patients was confirmed, which is not evident in non-TNBCs. Extending this knowledge, enhancement of immunologic activity in the TIME was found to be evident in only a subset of NAC-treated TNBC patients, but this activity correlates with improved RFS and OS. This activation is broad and does not appear confined to particular immunologic functions, likely representing the complexity involved in capturing immunologic activity at a single time point. However, the induction of cytolytic effector cell signatures in the TIME was particularly prognostic.

Interestingly, no immune-specific gene sets were significantly associated with poor outcome in TNBC patients after NAC. Immunologic activation in the TIME was also not accompanied by enhancement of TCR clonality. To determine if immunologic activation could be observed peripherally in patients treated with NAC, a series of functional and immunogenomic experiments were performed on CD8+ T cells isolated from PBMCs of patients undergoing NAC. The focus was specifically on PD-1^{HI} CD8+ T cells as these have been shown to be enriched for tumor-specific T cells⁸.

This population was confirmed to be more active and clonal by functional single-cell cytokine assays and TCR sequencing. In particular, a significant increase in cytolytic and inflammatory cytokines secreted by PD-1^{HI} CD8+ T cells were detected in two TNBC patients after chemotherapy, but not in two non-TNBC patients. A further characterization of PD-1^{HI} CD8+ cells by scRNAseq identified a population of cytolytic gene (e.g., GNLY, FGFBP2, GZMB)-expressing and checkpoint (e.g., LAG3, FCLR6 and substantially higher PDCD1)-expressing cells present in blood.

It was contemplated that these activated T cells in blood may be reflective of ongoing anti-tumor immunity, which could signify 1) the potential for ongoing tumor immunologic control and thus better outcome, or 2) persistent disease in the breast or micrometastatic compartment that ultimately leads to recurrence. Intriguingly, higher expression of a gene signature derived from these cytolytic cells in whole blood at the time of surgery was associated with higher disease burden (i.e. in those patients who did not receive NAC and those with RD who experienced disease recurrence within three years).

This finding was similar between TNBC and non-TNBC patients. Thus, peripheral cytotoxic activity, guarded by immune checkpoints, reflect ongoing micrometastatic and primary disease burden, and are useful for predicting disease recurrence and possibly immune checkpoint inhibitor benefit.

In studies presented in these examples, a molecular analysis of the TIME in response to NAC in 83 breast cancer patients is presented, specifically focusing on patients lacking a pCR, as these patients have worse outcomes. Like sTILs, changes in tumor immunity seem to be most prevalent in TNBC, often resulting in decreases in expression of immune-related genes. However, an upregulation of immune-related gene expression in tumors following NAC was associated with a strikingly improved outcome after surgery, specifically in TNBC. Of these genes, those involved in cytotoxic effector cells were among the most robustly associated with outcome. Furthermore, cytokines expressed by PD-1^{HI} CD8+ T cells in the peripheral blood were found to be increased dramatically in TNBC patients following NAC.

Analysis of TCR clonotypes infiltrating into tumors suggested that chemotherapy may preferentially increase the recruitment of new T cell clones into the tumor, rather than expanding the T cells already present. This effect was consistent with that observed in the peripheral blood, where PD-1^{HI} CD8+ T cells, while highly clonal compared to PD-1^{NEG} cells, did not substantially change in clonality during NAC; these observations reflect a lack of clonal expansion in response to NAC, as found in the TIME.

Assessment of peripheral blood represents a unique opportunity to monitor anti-tumor immunity through minimally-invasive means. Using scRNAseq, a population of cytolytic effector T cells were identified in blood that expressed elevated levels of exhaustion/checkpoint genes. A gene expression signature derived from this population was used to test the hypothesis that these highly cytolytic but potentially exhausted cells may be reflective of an ongoing disease process, and therefore a peripheral approximation of disease burden. This hypothesis was confirmed in a validation set of 60 patients and serves as a proof-of-principle for the use of this signature as a possible biomarker of outcome.

Importantly, there has been a paucity of studies looking at the effect of chemotherapy on peripheral blood²⁰, with little data on disease outcomes. These studies study provides a unique assessment and framework for an improved understanding of how chemotherapy alters anti-tumor immunity both in the TIME and the peripheral compartment. These data represent a unique opportunity to better understand patient populations most likely to benefit from the addition of immunotherapy to chemotherapy, particularly in the neoadjuvant setting.

Furthermore, the findings demonstrating the association of expression of key cytolytic and immune-activation genes in the peripheral blood with presence of residual disease and recurrence represent a possible biomarker platform.

The peripheral gene expression signature may identify high-risk populations with potentially exhausted T cells and either primary or micrometastatic disease who are likely to benefit from additional immunotherapeutic strategies.

### Example 15: Peripheral Blood in Breast Cancer Patients

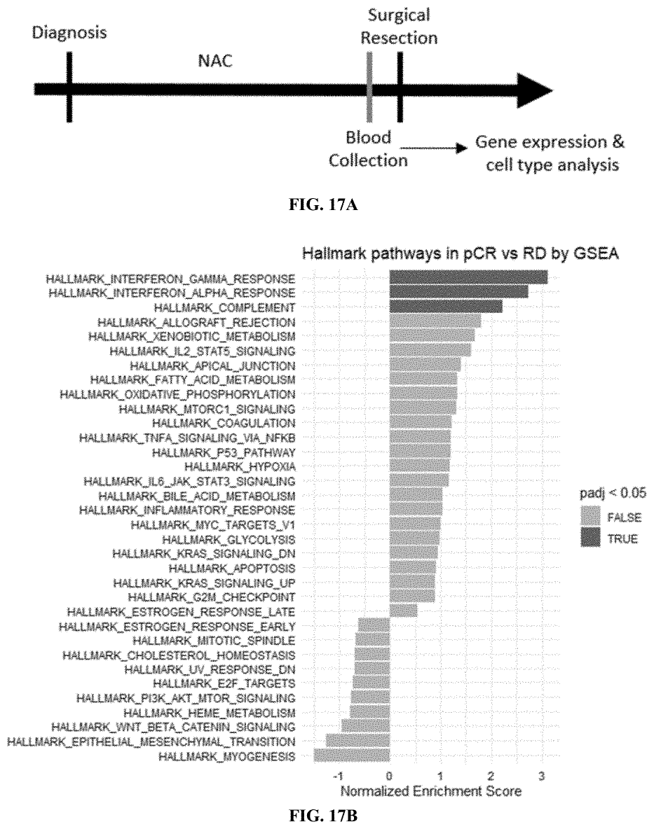

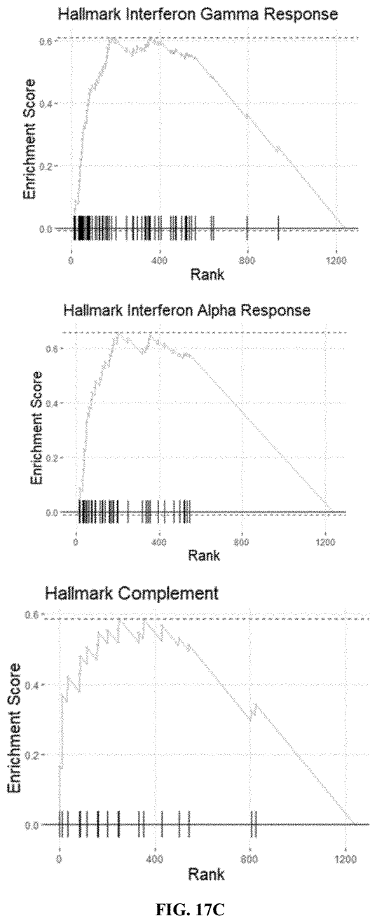

Peripheral blood in breast cancer patients was examined. With reference to FIG. 17A, blood was collected following neoadjuvant chemotherapy (NAC) and preceding surgical resection. Gene expression profiling and cell type analysis was used to predict outcome. Outcome at surgery (residual disease; RD or pathological complete response; pCR) was used as the primary outcome, as this is easily measurable and well-known to be associated with long term outcomes in breast cancer. In some cases, the patients were also able to stratified with residual disease based on whether or not they had a breast cancer recurrence in the three years following surgery (RD-R=residual disease with recurrence; RD-nR=residual disease with no recurrence). With reference to FIG. 17B, gene set enrichment analysis (GSEA) was used to identify groups of related genes that are differentially expressed in the blood of patients with pCR vs. RD. Three gene sets (Hallmark interferon gamma response, hallmark interferon alpha response and hallmark complement) were significantly upregulated in the blood of patients with pCR, compared to those with RD, after adjusting for multiple comparisons. With reference to FIG. 17C, enrichment plots show the upregulation of the three significant gene sets in patients with pCR.

### Example 16: Generation of Interferon (IFN)/Complement Score

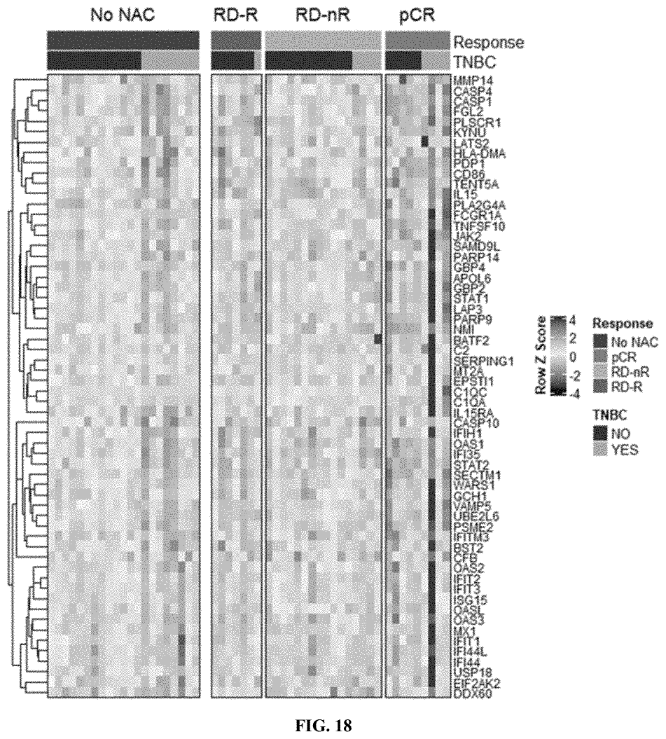

Using the GSEA findings, the leading edge genes from each of the three upregulated gene sets were taken. Leading edge genes are those most strongly enriched in the pCR patients vs the RD patients for each gene set. Sixty (60) unique genes were identified in the leading edge for the three enriched gene sets (genes and expression shown in heatmap in FIG. 18). Gene set scores are defined as the sums of the z-scores for each gene in the signature, divided by total number of genes in the signature.

### Example 17: Eight Gene Cytotoxic Signature and IFN/Complement Signature

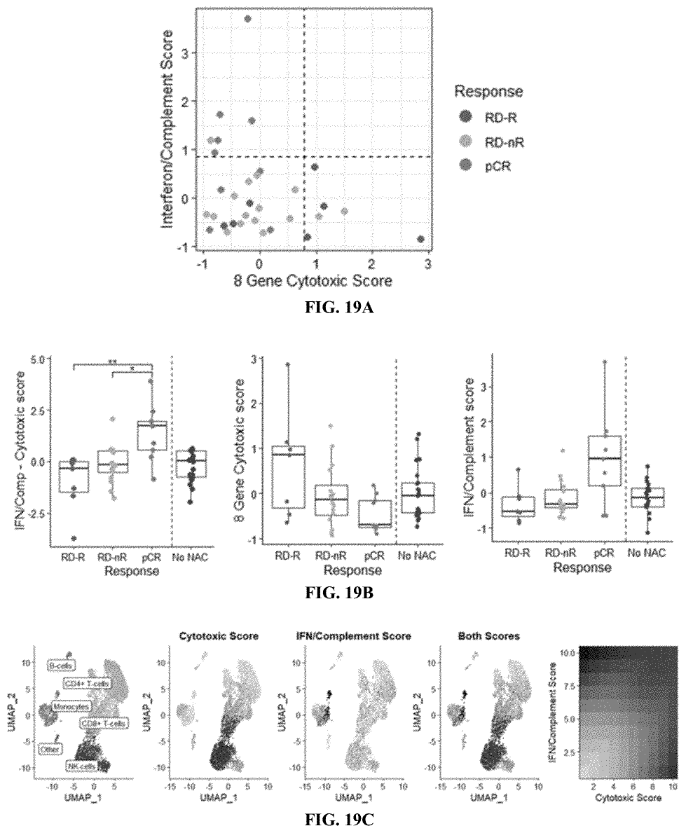

The 8 gene cytotoxic signature is defined as PDCD1+NKG7+LAG3+GZMH+GZMB+GNLY+FGFBP2−HLA-G. With reference to FIG. 19A, scores for the 8 gene cytotoxic signature and the IFN/complement score are shown. Samples highest for the 8 gene score are patients with residual disease, most with a breast cancer recurrence, and tend to be lowest for the IFN/complement score. Conversely, samples highest for the IFN/complement score are mostly patients with pCR and have low expression of the cytotoxic score. This demonstrates independence of the two signatures.

Turning now to FIG. 19B, a composite score of IFN/Complement signature minus cytotoxic signature predicts outcome in breast cancer patients. This composite score is highest in patients with pCR and lowest in patients with residual disease with recurrence. This composite score is a better predictor of outcome than either score alone.

The cytotoxic score is primarily expressed in peripheral blood CD8+ T cells and natural killer (NK) cells, while the IFN/Complement score is primarily expressed in monocytes. Single cell RNA sequencing was done on peripheral blood mononuclear cells (PBMCs) from two breast cancer patients, collected following NAC and prior to surgery). With reference to FIG. 19C, UMAP plots are shown for dimensionality reduction. Cell type annotations were done using singleR. Expression of each score is shown.

### Example 18: Monocytes in Predicting Outcomes

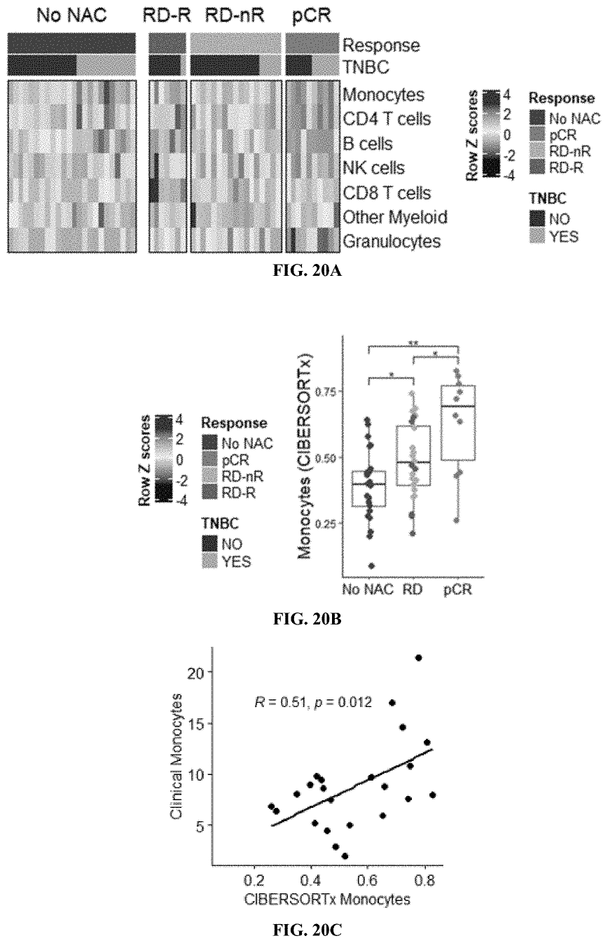

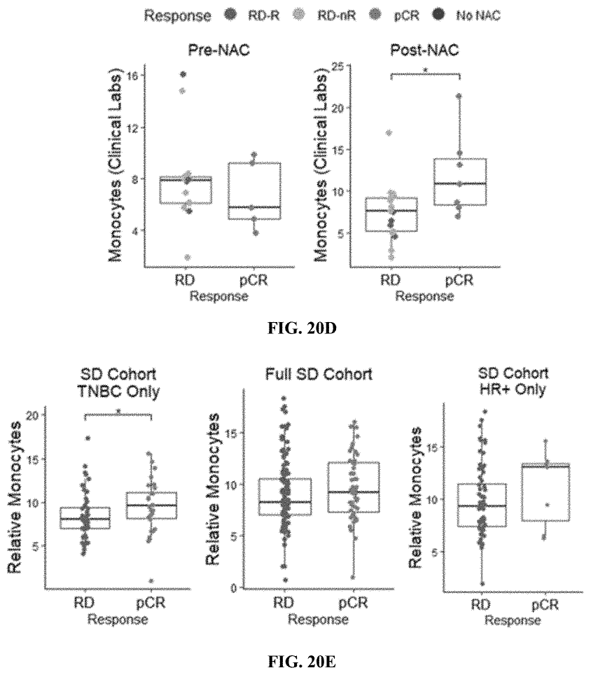

CIBERSORTx was used to deconvolute cell type abundance from bulk gene expression data. The heatmap in FIG. 20A shows row z-scores for each cell type. Monocytes are enriched in patients with a pCR. With reference to FIG. 20B, monocyte values, as determined by CIBERSORTx, are higher in patients with a pCR compared to those with residual disease or those who did not receive NAC.

For the same cohort of patients on whom peripheral blood gene expression profiling was conducted, clinically measured relative monocyte values were identified in the electronic medical record. With reference to FIG. 20C, clinically measured monocytes correlate strongly with CIBERSORTx inferred monocytes. With reference to FIG. 20D, post-NAC (in the same time interval as the gene expression) monocytes, but not pre-NAC monocytes, are higher in patients with pCR compared to those with RD.

The synthetic derivative, a de-identified medical record system, was used to identify additional breast cancer patients treated with NAC. With reference to FIG. 20E, in triple negative breast cancer (TNBC) patients in this cohort, relative monocytes, measured in the interval between NAC and surgery, were also significantly higher in patients with a pCR compared to those with RD. The same trend was observed in the larger cohort and in hormone receptor positive patients, but was not statistically significant. This may be due to heterogeneity of treatment for hormone receptor positive and HER2+ patients compared with patients with TNBC.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Schmid, P., et al. Atezolizumab and Nab-Paclitaxel in Advanced Triple-Negative Breast Cancer. The New England journal of medicine 379, 2108-2121 (2018).
2. Schmid, P., et al. LBA8 PRKEYNOTE-522: Phase III study of pembrolizumab (pembro)+chemotherapy (chemo) vs placebo (pbo)+chemo as neoadjuvant treatment, followed by pembro vs pbo as adjuvant treatment for early triple-negative breast cancer (TNBC). Annals of Oncology 30(2019).
3. Denkert, C., et al. Tumor-infiltrating lymphocytes and response to neoadjuvant chemotherapy with or without carboplatin in human epidermal growth factor receptor 2-positive and triple-negative primary breast cancers. Journal of clinical oncology: official journal of the American Society of Clinical Oncology 33, 983-991 (2015).
4. Loi, S., et al. Tumor-Infiltrating Lymphocytes and Prognosis: A Pooled Individual Patient Analysis of Early-Stage Triple-Negative Breast Cancers. Journal of clinical oncology: official journal of the American Society of Clinical Oncology 37, 559-569 (2019).
5. Luen, S. J., et al. Prognostic implications of residual disease tumor-infiltrating lymphocytes and residual cancer burden in triple-negative breast cancer patients after neoadjuvant chemotherapy. Annals of oncology: official journal of the European Society for Medical Oncology 30, 236-242 (2019).
6. Loi, S., et al. RAS/MAPK Activation Is Associated with Reduced Tumor-Infiltrating Lymphocytes in Triple-Negative Breast Cancer: Therapeutic Cooperation Between MEK and PD-1/PD-L1 Immune Checkpoint Inhibitors. Clin Cancer Res 22, 1499-1509 (2016).
7. Dieci, M. V., et al. Prognostic value of tumor-infiltrating lymphocytes on residual disease after primary chemotherapy for triple-negative breast cancer: a retrospective multicenter study. Annals of oncology: official journal of the European Society for Medical Oncology 25, 611-618 (2014).
8. Gros, A., et al. Prospective identification of neoantigen-specific lymphocytes in the peripheral blood of melanoma patients. Nature medicine 22, 433-438 (2016).
9. Rosshart, S., et al. Interaction of KLRG1 with E-cadherin: new functional and structural insights. European journal of immunology 38, 3354-3364 (2008).
10. Johnson, D. B., et al. Tumor-specific MHC-II expression drives a unique pattern of resistance to immunotherapy via LAG-3/FCRL6 engagement. JCI Insight 3(2018).
11. Han, M., et al. Metaplastic breast carcinoma: a clinical-pathologic study of 97 cases with subset analysis of response to neoadjuvant chemotherapy. Modern pathology: an official journal of the United States and Canadian Academy of Pathology, Inc 32, 807-816 (2019).
12. Kaminsky, A., Bhargava, R., McGuire, K. P. & Puhalla, S. Single institution experience with neoadjuvant chemotherapy for metaplastic breast cancer (MBC). Journal of Clinical Oncology 30, 1038-1038 (2012).
13. Bae, S. Y., et al. The prognoses of metaplastic breast cancer patients compared to those of triple-negative breast cancer patients. Breast cancer research and treatment 126, 471-478 (2011).
14. Aydiner, A., et al. Metaplastic Breast Carcinoma Versus Triple-Negative Breast Cancer: Survival and Response to Treatment. Medicine (Baltimore) 94, e2341 (2015).
15. Al-Hilli, Z., et al. Metaplastic breast cancer has a poor response to neoadjuvant systemic therapy. Breast cancer research and treatment 176, 709-716 (2019).
16. Ledderose, C., Heyn, J., Limbeck, E. & Kreth, S. Selection of reliable reference genes for quantitative real-time PCR in human T cells and neutrophils. BMC Res Notes 4, 427 (2011).
17. Dumont, C., et al. CD8(+)PD-1(−)ILT2(+) T Cells Are an Intratumoral Cytotoxic Population Selectively Inhibited by the Immune-Checkpoint HLA-G. Cancer immunology research 7, 1619-1632 (2019).
18. Carosella, E. D., Rouas-Freiss, N., Tronik-Le Roux, D., Moreau, P. & LeMaoult, J. HLA-G: An Immune Checkpoint Molecule. Adv Immunol 127, 33-144 (2015).
19. Lin, A. & Yan, W. H. Heterogeneity of HLA-G Expression in Cancers: Facing the Challenges. Front Immunol 9, 2164 (2018).
20. Foulds, G. A., et al. Immune-Phenotyping and Transcriptomic Profiling of Peripheral Blood Mononuclear Cells From Patients With Breast Cancer: Identification of a 3 Gene Signature Which Predicts Relapse of Triple Negative Breast Cancer. Front Immunol 9, 2028 (2018).
21. Balko, J. M., et al. Molecular profiling of the residual disease of triple-negative breast cancers after neoadjuvant chemotherapy identifies actionable therapeutic targets. Cancer Discov 4, 232-245 (2014).
22. Hendry, S., et al. Assessing Tumor-Infiltrating Lymphocytes in Solid Tumors: A Practical Review for Pathologists and Proposal for a Standardized Method from the International Immuno-Oncology Biomarkers Working Group: Part 2: TILs in Melanoma, Gastrointestinal Tract Carcinomas, Non-Small Cell Lung Carcinoma and Mesothelioma, Endometrial and Ovarian Carcinomas, Squamous Cell Carcinoma of the Head and Neck, Genitourinary Carcinomas, and Primary Brain Tumors. Adv Anat Pathol (2017).
23. Hendry, S., et al. Assessing Tumor-infiltrating Lymphocytes in Solid Tumors: A Practical Review for Pathologists and Proposal for a Standardized Method From the International Immunooncology Biomarkers Working Group: Part 1: Assessing the Host Immune Response, TILs in Invasive Breast Carcinoma and Ductal Carcinoma In Situ, Metastatic Tumor Deposits and Areas for Further Research. Adv Anat Pathol 24, 235-251 (2017).
24. Salgado, R., et al. The evaluation of tumor-infiltrating lymphocytes (TILs) in breast cancer: recommendations by an International TILs Working Group 2014. Ann Oncol 26, 259-271 (2015).
25. ImmunoMind. ImmunoMind Team. immunarch: An R Package for Painless Analysis of Large-Scale Immune Repertoire Data. (ImmunoMind Team, 2019).
26. Satija, R., Farrell, J. A., Gennert, D., Schier, A. F. & Regev, A. Spatial reconstruction of single-cell gene expression data. Nature biotechnology 33, 495-502 (2015).
27. Butler, A., Hoffman, P., Smibert, P., Papalexi, E. & Satija, R. Integrating single-cell transcriptomic data across different conditions, technologies, and species. Nature biotechnology 36, 411-420 (2018).
28. Korsunsky, I., et al. Fast, sensitive, and accurate integration of single cell data with Harmony. bioRxiv, 461954 (2018).
29. Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
30. Linderman, G. C., Zhao, J. & Kluger, Y. Zero-preserving imputation of scRNA-seq data using low-rank approximation. bioRxiv, 397588 (2018).
31. Gu, Z., Eils, R. & Schlesner, M. Complex heatmaps reveal patterns and correlations in multidimensional genomic data. Bioinformatics 32, 2847-2849 (2016).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of detecting expression of a combination of genes in a sample from a subject having breast cancer and who has received neoadjuvant chemotherapy (NAC), comprising:
(a) obtaining or having obtained a biological sample from the subject;
(b) detecting or having detected expression levels in the sample at least five genes of a first signature including the genes consisting of:
PDCD1, NKG7, LAG3, GZMH, GZMB, GNLY, FGFBP2, and HLA-DRB5.

2. The method of claim 1, and further comprising detecting or having detected expression levels in the sample at least ten genes of a second signature including the genes consisting of: SERPING1, IFIT3, IFI44L, IFI44, LAP3, FCGR1A, EPSTI1, IFIT2, TNFSF10, WARS1, IFITM3, MX1, MT2A, BATF2, IL15, IFIT1, STAT1, GBP4, ISG15, OAS3, JAK2, VAMP5, FGL2, PLSCR1, OASL, SAMD9L, USP18, SECTM1, APOL6, PLA2G4A, UBE2L6, CFB, PSME2, OAS2, STAT2, PARP14, CASP1, IFI35, HLA-DMA, GCH1, CD86, IL15RA, DDX60, LATS2, BST2, NMI, IFIH1, CASP4, EIF2AK2, PARP9, GBP2, TENT5A, OAS1, C1QC, C1QA, C2, KYNU, MMP14, PDP1, and CASP10.

3. The method of claim 1, and further comprising calculating a first signature score by adding the expression level (transcript count) of each of the genes selected for detection from PDCD1, NKG7, LAG3, GZMH, GZMB, GNLY, and FGFBP2; and subtracting the expression level (transcript count) of HLA-DRB5, if detected.

4. The method of claim 3, and further comprising identifying the subject as having a likelihood of residual disease (RD) when the first signature score is greater than a standardized control; or identifying the subject as having a likelihood of pathological complete response (pCR) when the first signature score is less than a standardized control.

5. The method of claim 4, and further comprising administering or recommending administration of additional chemotherapy prior to surgery and/or administration of additional chemotherapy after surgery when the subject is identified as having a likelihood of RD; or proceeding or recommending proceeding with surgery without administering additional chemotherapy when the subject is identified has having a likelihood of pCR.

6. The method of claim 1, and further comprising identifying the subject as having a likelihood of residual disease (RD) and/or cancer recurrence when there is an elevated level of each of the genes selected for detection from PDCD1, NKG7, LAG3, GZMH, GZMB, GNLY, and FGFBP2, and a reduced level of HLA-DRB5, if detected.

7. The method of claim 6, and further comprising administering or recommending administration of additional chemotherapy prior to surgery and/or administer additional chemotherapy after surgery.

8. The method of claim 1, and further comprising identifying the subject as having a likelihood of pathological complete response (pCR) when there is a reduced level of each of the genes selected for detection from PDCD1, NKG7, LAG3, GZMH, GZMB, GNLY, and FGFBP2, and an elevated level of HLA-DRB5, if detected.

9. The method of claim 8, and further comprising proceeding or recommending proceeding with surgery without administering additional chemotherapy.

10. The method of claim 1, wherein the subject has triple-negative breast cancer (TNBC).

11. The method of claim 1, wherein the biological sample is a peripheral blood sample.

12. The method of claim 11, wherein the biological sample is a buffy coat fraction of the whole peripheral blood, or purified immune cells from whole peripheral blood

13. The method of claim 1, wherein the biological sample is a sample comprising monocytes.

14. The method of claim 1, wherein the biological sample is a tumor sample or a sample obtained from the tumor-immune microenvironment.

15. The method of claim 1, wherein the biological sample is from a lymph node.

16. The method of claim 1, and further comprising extracting mRNA from the biological sample.

17. The method of claim 16, and further comprising measuring in the extracted mRNA the levels of mRNA of the at least five genes of the first signature.

18. The method of claim 17, and further comprising measuring in the extracted mRNA the levels of mRNA of normalization genes to control for the individual sample mRNA content.

19. The method of claim 18, wherein the normalization genes include at least two selected from the group consisting of: PTPRC, RPL13a, and TBP.

20. A method of detecting expression of a combination of genes in a sample from a subject having breast cancer and who has received neoadjuvant chemotherapy (NAC), comprising:
(a) obtaining or having obtained a biological sample from the subject;
(b) detecting or having detected expression levels in the sample at least ten genes of a second signature including the genes consisting of:
SERPING1, IFIT3, IFI44L, IFI44, LAP3, FCGR1A, EPSTI1, IFIT2, TNFSF10, WARS1, IFITM3, MX1, MT2A, BATF2, IL15, IFIT1, STAT1, GBP4, ISG15, OAS3, JAK2, VAMP5, FGL2, PLSCR1, OASL, SAMD9L, USP18, SECTM1, APOL6, PLA2G4A, UBE2L6, CFB, PSME2, OAS2, STAT2, PARP14, CASP1, IFI35, HLA-DMA, GCH1, CD86, IL15RA, DDX60, LATS2, BST2, NMI, IFIH1, CASP4, EIF2AK2, PARP9, GBP2, TENT5A, OAS1, C1QC, C1QA, C2, KYNU, MMP14, PDP1, and CASP10; and
(c) calculating a second signature score by adding the expression level (transcript count) of each of the genes selected for detection, and identifying the subject as having a likelihood of residual disease (RD) when the second signature score is less than a standardized control; or identifying the subject as having a likelihood of pathological complete response (pCR) when the second signature score is greater than a standardized control; or
identifying the subject as having a likelihood of residual disease (RD) and/or cancer recurrence when there is a reduced level of each of the genes selected for detection, or identifying the subject as having a likelihood of pathological complete response (pCR) when there is an elevated level of each of the genes selected for detection; and
(d) administering or recommending administration of additional chemotherapy prior to surgery and/or administer additional chemotherapy after surgery when the subject is identified as having a likelihood of RD; or proceeding or recommending proceeding with surgery without administering additional chemotherapy when the subject is identified has having a likelihood of pCR.


SEQUENCE LISTING (24 sequences)

SEQ ID NO: 1 - DNA, 100 bp, Artificial Sequence
  Synthesized
cttccccgaggaccgcagccagcccggccaggactgccgcttccgtgtcacacaactgcccaacgggcgtgacttccacatgagcgtggtcagggcccgg

SEQ ID NO: 2 - DNA, 70 bp, Artificial Sequence
  Synthesized
tgacacggaagcggcagtcctggccgggctggctgcctggagtttatgtattgccaacgagtttgtcttt

SEQ ID NO: 3 - DNA, 68 bp, Artificial Sequence
  Synthesized
cgaaagccatgacctccgatcactcctgaccacgctcatgtggaagtcacgcccgttgggcagttgtg

SEQ ID NO: 4 - DNA, 100 bp, Artificial Sequence
  Synthesized
ctgtggcggtccccgtcctggctatgaaaccttgtgagcagaaggcaagagcggcaagatgagttttgagcgttgtattccaaaggcctcatctggagcc

SEQ ID NO: 5 - DNA, 79 bp, Artificial Sequence
  Synthesized
tcttgccttctgctcacaaggtttcatagccaggacggggaccgcgaacctaactcctcgctacattcctattgttttc

SEQ ID NO: 6 - DNA, 75 bp, Artificial Sequence
  Synthesized
cgaaagccatgacctccgatcactcggctccagatgaggcctttggaatacaacgctcaaaactcatcttgccgc

SEQ ID NO: 7 - DNA, 100 bp, Artificial Sequence
  Synthesized
cttttggtgactggagcctttggctttcacctttggagaagacagtggcgaccaagacgattttctgccttagagcaagggattcaccctccgcaggctc

SEQ ID NO: 8 - DNA, 85 bp, Artificial Sequence
  Synthesized
cgccactgtcttctccaaaggtgaaagccaaaggctccagtcaccaaaagcagataaggttgttattgtggaggatgttactaca

SEQ ID NO: 9 - DNA, 72 bp, Artificial Sequence
  Synthesized
cgaaagccatgacctccgatcactccctgcggagggtgaatcccttgctctaaggcagaaaatcgtcttggt

SEQ ID NO: 10 - DNA, 100 bp, Artificial Sequence
  Synthesized
aaaaaagggacacctccaggagtctacatcaaggtctcacacttcctgccctggataaagagaacaatgaagcgcctctaacagcaggcatgagactaac

SEQ ID NO: 11 - DNA, 85 bp, Artificial Sequence
  Synthesized
ggcaggaagtgtgagaccttgatgtagactcctggaggtgtcccttttttccaatttggttttactcccctcgattatgcggagt

SEQ ID NO: 12 - DNA, 75 bp, Artificial Sequence
  Synthesized
cgaaagccatgacctccgatcactcgttagtctcatgcctgctgttagaggcgcttcattgttctctttatccag

SEQ ID NO: 13 - DNA, 100 bp, Artificial Sequence
  Synthesized
acactacaagaggtgaagatgacagtgcaggaagatcgaaagtgcgaatctgacttacgccattattacgacagtaccattgagttgtgcgtgggggacc

SEQ ID NO: 14 - DNA, 85 bp, Artificial Sequence
  Synthesized
gattcgcactttcgatcttcctgcactgtcatcttcacctcttgtagtgtcacaattctgcgggttagcaggaaggttagggaac

SEQ ID NO: 15 - DNA, 75 bp, Artificial Sequence
  Synthesized
cgaaagccatgacctccgatcactcggtcccccacgcacaactcaatggtactgtcgtaataatggcgtaagtca

SEQ ID NO: 16 - DNA, 100 bp, Artificial Sequence
  Synthesized
tgccggctcctcgcttcctcgatccagaatccactctccagtctccctcccctgactccctctgctgtcctcccctctcacgagaataaagtgtcaagca

SEQ ID NO: 17 - DNA, 79 bp, Artificial Sequence
  Synthesized
ggagggagactggagagtggattctggatcgaggaagcgaggagcatcctcttcttttcttggtgttgagaagatgctc

SEQ ID NO: 18 - DNA, 75 bp, Artificial Sequence
  Synthesized
cgaaagccatgacctccgatcactctgcttgacactttattctcgtgagaggggaggacagcagagggagtcagg

SEQ ID NO: 19 - DNA, 100 bp, Artificial Sequence
  Synthesized
ctttctggagtttgcagagttcagcaatatgatagggaacaggtgctgatgggcccaagagtgacaagcatacacaactacttattatctgtagaagttt

SEQ ID NO: 20 - DNA, 85 bp, Artificial Sequence
  Synthesized
atcagcacctgttccctatcatattgctgaactctgcaaactccagaaagcctcaagacctaagcgacagcgtgaccttgtttca

SEQ ID NO: 21 - DNA, 75 bp, Artificial Sequence
  Synthesized
cgaaagccatgacctccgatcactcaaacttctacagataataagtagttgtgtatgcttgtcactcttgggccc

SEQ ID NO: 22 - DNA, 100 bp, Artificial Sequence
  Synthesized
gagtgtcatttcttcaacgggacggagcgggtgcggttcctgcacagagacatctataaccaagaggaggacttgcgcttcgacagcgacgtgggggagt

SEQ ID NO: 23 - DNA, 70 bp, Artificial Sequence
  Synthesized
cgcacccgctccgtcccgttgaagaaatgacactccaaagacgcctatcttccagtttgatcgggaaact

SEQ ID NO: 24 - DNA, 60 bp, Artificial Sequence
  Synthesized
cgaaagccatgacctccgatcactccctcctcttggttatagatgtctctgtgcaggaac